(19) United States
(12) Patent Application Publication
(10) Pub. No.: US 2010/0150938 A1
(43) Pub. Date: Jun. 17, 2010

(54) METHODS AND COMPOSITIONS FOR REDUCING INFLAMMATION AND TREATING INFLAMMATORY DISORDERS

(75) Inventors: Eicke Latz, Bonn (DE); Douglas Golenbock, Wellesley, MA (US); Kenneth L. Rock, Chestnut Hill, MA (US); Veit Hornung, Bonn (DE); Annett Halle, Berlin (DE)

Correspondence Address: LAHIVE & COCKFIELD, LLP;FLOOR 30, SUITE 3000, ONE POST OFFICE SQUARE, BOSTON, MA, 02109, US

(73) Assignee: UNIVERSITY OF MASSACHUSETTS, Boston, MA (US)

(21) Appl. No.: 12/497,357

(22) Filed: Jul. 2, 2009

Related U.S. Application Data
(60) Provisional application No. 61/078,156, filed on Jul. 3, 2008.
(60) Provisional application No. 61/078,440, filed on Jul. 6, 2008.

(51) Int. Cl. A61K 39/395; A61K 38/02; A61K 31/7052; A61P 29/00

(52) U.S. Cl. 424/158.1; 514/2; 514/44 A

(57) ABSTRACT

Methods of treating an inflammatory disorder and inhibiting inflammation by administering an inhibitor of a pH-activated protease are provided.

### CROSS REFERENCE TO RELATED APPLICATIONS

This application is related and claims priority to U.S. provisional application Ser. No. 61/078,440 filed Jul. 6, 2008; U.S. provisional application Ser. No. 61/078,156 filed Jul. 3, 2008. The entire contents of each of the foregoing applications are incorporated herein by this reference.

### BACKGROUND OF THE INVENTION

Inflammation of cells and tissues may be triggered by exposure to a variety of environmental and internal stimuli resulting in both acute and chronic inflammatory conditions. For example, the intake of exogenous pathogenic particulates (e.g., asbestos, silica) and microbes leads to rapid recruitment of immune cells and production of cytokines and other factors that induce inflammation in the exposed tissues. Inflammatory responses are also triggered by the release or accumulation of endogenous danger signals (DAMPs) (e.g., uric acid, galectins, thoredoxin, ATP) in response to necrotic cell death resulting from trauma or other pathological conditions.

The proinflammatory cytokine interleukin-1β (IL-1β) has been reported to play a major role in the onset of inflammation. Mature IL-1β is produced by cleavage of the inactive pro-IL-1β precursor by caspase-1, the activity of which is tightly controlled by cytosolic multi-protein complexes called inflammasomes (Dinarello, C. A. Immunity 26, 383-385, 2007). The NACHT-, LRR-, and PYD domain-containing proteins (NALP) are central components of the inflammasome that associate with the adaptor protein apoptosis-associated speck-like protein (ASC), which in turn recruits pro-inflammatory-caspase precursors, such as pro-caspase-1. Recently it has been reported that inflammasomes containing NALP3 can be activated by bacterial toxins and pathogen-associated molecular patterns (PAMPs), as well as endogenous stress-associated danger signals (e.g., MSU, CPPD, ATP) (Martinon, F., et al. Nature 440, 237-241 (2006);. Mariathasan, S. et al. Nature 440, 228-232 (2006); , Petrilli, V., et al., Curr. Opin. Immunol. 19, 615-622 (2007)). The NALP3 inflammasome has also been implicated in the pathological increase of IL-1β production in autoinflammatory syndromes, such as Muckle-Wells syndrome (Agostini et al., Immunity 20, 319 (2004)), inflammatory processes, such as gout and pseudogout (Marinon et al., supra), and pulmonary inflammatory diseases that are linked to pathogenic air pollutants (Dostert et al., Science 320, 674-677, 2008).

However, while inflammation is a hallmark of exposure to pathogenic particulates, microbial pathogens and DAMPs, the mechanism of NALP3 inflammasome activation and IL-1β production in response to these stimuli remains unclear. It has been proposed that the activating molecules could directly interact with NALP3 after entering the cell, or that they could modify one or more membrane-associated proteins (Dostert et al., supra). Recent studies have also implicated reactive oxygen species (ROS) with signaling pathways associated with inflammation (Shukla et al., Free Radic. Biol. Med. 34, 117, 2003) and inflammasome activation (Petrilli et al. Cell Death Differ 14, 1583, 2007). Accordingly, determining the factors that play a significant role in NALP3 inflammasome activation and IL-1β release in response to exogenous and endogenous pathogenic stimuli is of great clinical import in order to develop novel therapeutic strategies for treating inflammatory disorders.

### SUMMARY OF THE INVENTION

The present invention is based, at least in part, on the unexpected discovery that activation of the NALP3 inflammasome and subsequent release of IL-1β is the result of lysosomal acidification and the activity of pH-activated proteases (e.g., cathepsins including cathepsins B and L).

Accordingly, in one aspect, the invention feature a methods for treating an inflammatory disorder induced by particulate matter comprising administering to a subject in need thereof an effective amount of an inhibitor of a pH-activated protease, such that the particulate-induced inflammatory disorder is treated.

In a related aspect, the invention features a method of inhibiting particulate-induced caspase-1 activation comprising administering to a subject in need thereof an effective amount of an inhibitor of a pH-activated protease, such that the particulate-induced caspase-1 activation is inhibited.

In another related aspect, the invention features a method of inhibiting particulate-induced NALP3-ASC-dependent caspase-1 activation comprising administering to a subject in need thereof an effective amount of an inhibitor of a pH-activated protease, such that the particulate-induced NALP3-ASC-dependent caspase-1 activation is inhibited.

In some embodiments of these aspects of the invention, the particulate matter is a crystal or a fiber. In certain embodiments, the particulate matter is monosodium urate (MSU), aluminum salt, silica, calcium pyrophosphate dehydrate (CPPD), cholesterol, beta amyloid, asbestos or a nonasbestiform mineral fiber.

In other embodiments, the particulate matter is protein aggregates or necrotic cellular debris. In certain embodiments, the particulate matter is minimally modified LDL, aggregated peptides and aggregated proteins (e.g., aggregated alpha-synuclein, copper-zinc superoxide dismutase, or prion containing protein aggregates). In one embodiment, the inflammatory disorder is induced by a kidney stone.

In another aspect, the invention features a method for treating an inflammatory disorder related to NALP3-ASC dependent caspase-1 activation by administering to a subject in need thereof an effective amount of an inhibitor of a pH-activated protease, such that the NALP3-ASC dependent caspase-1 activation-induced inflammatory disorder is treated.

In certain embodiments of all aspects of the invention, the pH-activated protease is a lysosomal protease. In other embodiments, the pH-activated protease is a cathepsin. In other embodiments the cathepsin is selected from the group consisting of cathepsin B, L, H and S. In one embodiment, the cathepsin is cathepsin B. In one embodiment, the cathepsin is cathepsin L.

In some embodiments of the invention, the inflammatory disorder is a pulmonary disorder, including but not limited to acute lung injury, acute respiratory distress syndrome, asthma, silicosis, pneumonoconiosis, fiber-induced pulmonary fibrosis, asbestosis, chronic obstructive pulmonary disease, chronic bronchitis, emphysema and bronchiectasis.

In some embodiments, the inflammatory disorder is acute or chronic joint inflammation. In certain embodiments, the joint inflammation is gout or pseudogout or arthritis.

In one embodiment, the inflammatory disorder is a cardiovascular disorder such as atherosclerosis or reperfusion injury.

In one embodiment, the inflammatory disorder is amyloidosis.

In one embodiment, the inflammatory disorder is associated with transplant rejection (e.g., acute transplant rejection, chronic rejection or chronic allograft vasculopathy).

In one embodiment, the inflammatory is a chronic non-healing of physical injury.

In one embodiment, the inflammatory disorder liver inflammation.

In other embodiments, the inflammatory is an autoimmune disease including, but not limited to systemic lupus erythematosus, rheumatoid arthritis or vasculitis, including immune complex vasculitis.

In other embodiments, the inflammatory disorder is a neurodegenerative disease including Parkinson's disease, Alzheimer's disease, Amyotrophic Lateral Sclerosis and Creutzfeldt-Jakob disease.

In other embodiments, the inflammatory disorder a periodic fever syndrome including, but not limited to Familial Mediterranean fever; TNF receptor 1-associated periodic syndrome; Hyper-IgD syndrome; Periodic fevers with Aphthous stomatitis, Pharyngitis and Adentitis syndrome; pyogenic sterile arthritis, pyoderma gangrenosum and acne syndrome; Blau syndrome; and a cryopyrin-associated periodic syndrome (e.g., familial cold autoinflammatory syndrome, Muckle-Wells syndrome and neonatal onset multisystem inflammatory disorder).

In one embodiment, the inflammatory disorder treated and/or inhibited by methods of the invention is a blockage of the ureter.

The invention also features kits containing pharmaceutical compositions containing inhibitors of pH-activated protease inhibitors.

Additional embodiments of the invention are described in the detailed description and Examples described herein.

### BRIEF DESCRIPTION OF THE FIGURES

FIG. 1(a)-(c) shows the effects of silica on the release of mature IL-1β and the activation of caspase-1 in human PBMCs: (a) Human PBMCs were primed with LPS (25 pg/ml) or left untreated for 3 h and subsequently stimulated with silica crystals or controls. After 6 h, supernatants were assessed for IL-1β production by ELISA and Western blot. ELISA data of four independent donors are depicted (upper panel) and Western blot analysis of one representative donor is shown (lower panel). (b) LPS-primed human PBMCs were stimulated with either silica crystals, MSU crystals, ATP or transfected with dAdT. 6 h after stimulation, supernatants were analyzed for IL-1β by ELISA and assessed for matured IL-1β or activated caspase-1 by Western blot. Data of one representative donor out of three are depicted. (c) Human LPS-primed PBMCs were stimulated with silica crystals in the presence or absence of the caspase-1 inhibitor z-YVAD (10 □M). After 6 h, supernatants were assessed for IL-1β by ELISA and Western blot. Mean values (+SD) of two donors are depicted as fold increase (ELISA) and Western blot data of one representative donor are shown. FIG. 1(d)-(e) shows the ability of silica to mediate a neutrophil influx in wild type, MyD88^{-/-} and IL-1 receptor^{-/-} mice. (d) Silica crystals (200 μg/ mouse) were orotracheally instilled into wild-type mice, MyD88/TRIF-double-deficient mice or IL-1R-deficient mice. 16-18 h after instillation, neutrophil counts were monitored in the lung lavage by FACS. (e) In addition, wild-type mice, MyD88-/TRIF-double-deficient mice or IL-1R-deficient mice were orotracheally challenged with zymosan (50 μg/mouse) and processed as in (a).

FIG. 2 shows the effects of silica and MSU on IL-1β release and caspase-1 activation in wild type, NALP3^{-/-} and ASC^{-/-} mouse macrophages (panels a & b), as well as the effects of uricase on percent maximum IL-1β response (panel c). (a) Bone marrow-derived macrophages of wild-type mice, NALP3-deficient mice or ASC-deficient mice were primed with LPS for 3 h and subsequently stimulated with either silica crystals, MSU crystals, ATP or transfected with dAdT. 6 h after stimulation, supernatants were analyzed for IL-1β by ELISA (supernatants). To assess intracellular pro-IL-1β, cell lysates from primed, but unstimulated macrophages were assessed for IL-1β by ELISA (lysate). (b) In addition, supernatants were assessed for activated caspase-1 by Western blot. (c) LPS-primed mouse macrophages were stimulated with silica or MSU crystals as indicated in absence or presence of uricase as shown. Supernatants were assessed for IL-1β by ELISA).

FIG. 3 shows the effects of cytochalasin D on crystal-mediated IL-1β release and the effects of silica, MSU, ATP and dAdT on IL-1β release and caspase-1 activation (for silica and MSU) in wild-type, gp91Phox^{-/-} and gp91Phox^{+/+}/IPAF^{-/-} mice. (a) Human LPS-primed PBMCs were treated with cytochalasin D in ascending doses and subsequently stimulated with silica crystals, MSU crystals or ATP. IL-1β release was measured by ELISA 6 h after stimulation. (b) B6-MCLs were stimulated for 2 h with silica crystals in the presence or absence of cytochalasin D (2.5 μM). Cells were then membrane stained with fluorescent choleratoxin (red), nuclei stained with Hoechst dye (blue) and analyzed for crystal uptake (green) using confocal microscopy. (c) B6-MCLs were incubated with silica crystals as in (b) and phagocytosed silica crystals was analyzed for their length and the fractional distribution of crystal sizes is shown from phagocytosed crystals of 10 representative cells. (d and e) Bone marrow-derived macrophages of wild-type mice, gp91Phox-deficient mice or IPAF-deficient mice (as a mixed background control) were primed with LPS for 3 h and subsequently stimulated with either silica crystals, MSU crystals, ATP or transfected with dAdT. 6 h after stimulation, supernatants were analyzed for IL-1β by ELISA (d) and assessed for activated caspase-1 by Western blot (e). (f) B6-MCLs were stimulated for 2 h with MSU crystals in the presence or absence of cytochalasin D (2.5 μM). Cells were then membrane stained with fluorescent choleratoxin (red), nuclei stained with Hoechst dye (blue) and analyzed for crystal uptake (green) using confocal microscopy.

FIG. 4 shows the effects of crystal phagocytosis on lysosomal stability. (a) B6-MCLs were incubated with 10 μg/ml DQ-ovalbumin (red) alone or together with silica crystals (green) for 60 min, surface stained with fluorescent choleratoxin (blue) and analyzed by confocal microscopy. b(b) B6-MCLs were incubated with silica crystals (green) for 60 min or left untreated, fixed, permeabilized (saponin 0.01%) and stained with fluorescent choleratoxin (red), Hoechst dye (blue) and analyzed by confocal microscopy. (c) B6-MCLs and NALP3-KO-MCLs were stained with acridine orange and subsequently treated with silica crystals as indicated. 3 h after treatment cells were analyzed by flow cytometry for lysosomal acridine orange fluorescence. (d) B6-MCLs were treated with bafilomycin as indicated and stained with lysosensor green (1 μM) immediately prior to flow cytometry. (e) B6-MCLs were incubated with DQ-ovalbumin in the presence or absence of bafilomycin (250 nM) for 60 min and subjected to FACS analysis. (f) LPS-primed B6-MCLs were treated with bafilomycin or left untreated and subsequently stimulated with silica crystals or ATP. IL-1β release was measured by ELISA 6 h after stimulation

FIG. 5 shows the effects of a cathepsin B on silica-mediated IL-1β production. (a) LPS-primed B6-MCLs were treated with either cathepsin B inhibitor (CA-074-Me, 10 μM) or left untreated and subsequently stimulated with silica crystals, ATP or transfected with dAdT. IL-1β release was measured by ELISA 6 h after stimulation. Data from one representative experiment out of two are depicted. (b) Bone marrow-derived macrophages of wild-type mice or NALP3-deficient mice were primed with LPS for 3 h and subsequently stimulated with either silica crystals, MSU crystals, ATP or transfected with dAdT. 6 h after stimulation, supernatants were analyzed for cathepsin B by Western blot. (c) B6-MCLs were incubated with silica crystals (pink) for 3 h or left untreated. Subsequently, cells were stained with fluorescent probes for activated caspase-1 (green) and activated cathepsin B (red) for one additional hour and then surface stained with fluorescent choleratoxin (blue) and analyzed by confocal microscopy. See also FIG. 26 below.

FIG. 6 shows the effects of alum on lysosomal stability and the NALP3 inflammasome. (a) Human PBMCs were primed with LPS (25 pg/ml) or left untreated for 3 h and subsequently stimulated with alum in ascending doses. After 6h, supernatants were assessed for IL-1β production by ELISA and Western blot. ELISA data of four independent donors are depicted (upper panel) and Western blot analysis of one representative donor is shown (lower panel). (b) Bone marrow-derived macrophages of wild-type mice, NALP3-deficient mice or ASC-deficient mice were primed with LPS for 3 h and subsequently stimulated with alum (500 μg/ml) and supernatants were analyzed for IL-1β by ELISA. (c) Alum (100 μg) was injected i.p. into wild-type mice (n=5) or IL1R-deficient mice (n=5), whereas PBS served as a control in wild-type mice (n=3). 16-18 h after injection neutrophil counts were monitored in the peritoneal lavage by FACS. (d) B6-MCLs were incubated with 10 μg/ml DQ-ovalbumin (red) alone or together with alum (green) for 60 min, surface stained with fluorescent choleratoxin (blue) and analyzed by confocal microscopy. (e) B6-MCLs were stained with acridine orange and subsequently treated with alum (blue) as indicated. (f) B6-MCLs were incubated with alum (pink) for 3 h or left untreated. Subsequently, cells were stained with fluorescent probes for activated caspase-1 (green) and activated cathepsin B (red) for one additional hour and then surface stained with fluorescent choleratoxin (blue) and analyzed by confocal microscopy. (g) LPS-primed bone marrow-derived macrophages were treated with either cathepsin B inhibitor (CA-074-Me, 10 μM), bafilomycin (250 nM) or left untreated and subsequently stimulated with alum. IL-1β release was measured by ELISA 6 h after stimulation. (h) LPS-primed bone marrow-derived macrophages were treated with either alum or MSU in the presence of ascending doses of uricase. IL-1β release was measured by ELISA 6 h after stimulation. Data were normalized to the condition without uricase.

FIG. 7 shows the effects of lysosomal rupture on the NALP3 inflammasome. (a) B6-MCLs were incubated in the presence of fluorescent dextran (red) for 30 min and were left untreated or were subsequently treated using hypertonic and hypotonic solutions to induce lysosomal rupture. (b) Bone marrow-derived macrophages of wild-type mice or NALP3-deficient mice were treated as in (a) in the presence or absence of cathepsin B inhibitor (CA-074-Me, 10 or 2 μM). In addition, ATP or dAdT were used as controls. 5 h after stimulation supernatants were analyzed for activated caspase-1. (c) B6-MCLs were labeled with acridine orange (upper panel) or incubated in the presence of fluorescent dextran (red; lower panel) and incubated with Leu-Leu-OMe (1000 μM). 3 h after incubation cells were analyzed by confocal microscopy. (d) LPS-primed B6-MCLs were incubated with Leu-Leu-OMe (1000 or 2000 μM), silica crystals (250 μg/m1), ATP or dAdT. IL-1β release was measured by ELISA 6 h after stimulation. (e) Bone marrow-derived macrophages of wild-type mice, NALP3-deficient mice or ASC-deficient mice were primed with LPS and subsequently stimulated with Leu-Leu-OMe (500 or 1000 μM) and ATP in the presence or absence of cathepsin B inhibitor (CA-074-Me, 10 μM) or bafilomycin (250 nM) (upper panel). In addition cells were stimulated with silica crystals or dAdT in the presence or absence of cathepsin B inhibitor (CA-074-Me, 10 μM) or bafilomycin (250 nM) (lower panel).

FIG. 8 (a) shows the response to inflammasome stimuli in immortalized murine macrophage cells lines. LPS-primed B6-MCLs, NALP3-KO-MCLs and ASC-KO-MCLs were stimulated with ATP, silica crystals, MSU crystals or transfected with dAdT. Release of activated caspase-1 was assessed by Western blot.

FIG. 8(b) shows a diagram of the technique of laser scanning confocal microscopy combined with reflection microscopy. In standard laser scanning confocal microscopy, the light path is set up to separate the laser light from the emission of the fluorophore to be studied. When laser scanning confocal microscopy is combined with reflection microscopy, a portion of the laser light that is reflected from crystal structures is collected by a separate detector. Since reflection and fluorescence emission of an excited fluorophore derive from the same confocal plane (as determined by the pinholes), one can achieve accurate spatial resolution of crystal localization in relation to subcellular structures.

FIG. 9(a) shows the effects of silica crystal uptake on lysosomal integrity. B6-MCLs were incubated with A647-Dextran for 30 min and then either left untreated or stimulated with silica crystals (250 μg/ml). 90 min after incubation cells were analyzed by confocal microscopy. Representative visual fields are depicted.

FIG. 9(b)-(c) demonstrates the use of acridine orange as a technique to study lysosomal distribution. (b) B6-MCLs were labeled with acridine orange and subsequently stimulated with silica crystals. 60 min after stimulation cells were analyzed by confocal microscopy. (c) AO absorbs light at approximately 490 nm and emits between 510 and 560 nm for green fluorescence (bound DNA) and between 600 and 650 nm for red fluorescence (lysosomes). A lambda scan is shown for AO staining of B6-MCLs with the two characteristic peaks for DNA-bound AO and lysosomal AO.

FIG. 10 shows the induction of caspase-1 dependent release of IL-1β by fibrillar β-Amyloid. (a-b) Wild type primary mouse microglia and wild type immortalized mouse microglia were stimulated with fibrillar Aβ (10 μM), reverse Aβ (10 μM), or left untreated. Aβ induced time-dependent release of IL-1β, assessed by ELISA in supernatants. Values represent means ±s.e.m. (c) Aβ activates caspase-1 in microglia. Wild type immortalized microglial cells were incubated with Aβ (10 μM), revAβ (10 μM), ATP (1 mM), or left untreated. After 4 h, cells were incubated with FAM-YVAD-fmk, and fluorescence of activated caspase-1 (green) was assessed using confocal microscopy (upper panel) or flow cytometry (lower panel). Cellular membranes were stained with choleratoxin subunit b (red). Scale bar=10 μm. (d) LPS-primed wild type bone marrow-derived macrophages were stimulated with Aβ (1, 5 and 10 μM), revAβ (10 μM), ATP (5 mM) or transfected with dAdT (1.6 μg). Supernatants were assessed for caspase-1 cleavage (p10) by Western blot analysis. (e) Wild type immortalized microglial cells were stimulated with fibrillar Aβ in the presence of increasing amounts of the caspase-1 inhibitor z-YVAD-fmk. Analysis of IL-1β release into supernatants after 6 h using ELISA revealed significant attenuation of IL-1β production by caspase-1 inhibition.

FIG. 11 shows the effects of fibrillar β-Amyloid on the NALP3 inflammasome. (a) Aβ induces activation of ASC. Wild type immortalized microglial cells were stably transduced with a fusion protein of ASC and cyan fluorescence protein (CFP), and stimulated with revAβ (10 μM), Aβ (10 μM), or ATP (1 mM) for 4 h in duplicate after priming with LPS. ASC activation was indicated by appearance of strongly fluorescent clusters of ASC-CFP (triangles, red), as assessed with confocal microscopy. Cell membranes were stained with fluorescent choleratoxin subunit b (Ctb, green). Scale bars=10 μm. (b) Quantification of images representatively shown in (a). 5 random fields per duplicate were imaged and quantified. ASC-CFP clusters formed upon stimulation with Aβ and ATP, but not after stimulation with revAβ or in untreated cells (mean±s.e.m.). (c) LPS-primed bone marrow-derived macrophages from wild type, NALP3^{-/-} and ASC^{-/-} mice were stimulated with either Aβ (1, 5 and 10 μM), revAβ (10 μM), ATP (5 mM), or transfected with dAdT (200 ng). Analysis of IL-1β in the supernatants 6 h after stimulation by ELISA revealed that NALP3^{-/-} and ASC^{-/-} cells failed to produce IL-1β after stimulation with Aβ. As controls, strong IL-1β release was detected in NALP3^{-/-} macrophages after transfection with dAdT.

FIG. 12 shows the effects of β-Amyloid phagocytosis on IL-1β release and lysosomal integrity. (a) In wild type immortalized microglia, cytochalasin D, an inhibitor of Aβ phagocytosis, dose-dependently inhibited IL-1β release, as assessed by IL-1β quantification in the supernatants using ELISA. ATP-dependent IL-1β release was not affected by Cytochalasin D. (b) Confocal microscopy of Aβ phagocytosis in immortalized microglia. Cells were incubated with FITC-labeled Aβ (10 μM) for 4 h and processed for immunocytochemistry. Cell membranes were visualized with fluorescent choleratoxin subunit b (Ctb) and lysosomes were stained using antibodies against LAMP-1. Scale bar=10 μm. (c) LAMP-1 positive Aβ-containing lysosomes adopted an enlarged appearance (arrows), indicating lysosomal swelling. Insets show z-series through indicated lines. Scale bar=10 μm. (d) Quantification of changes in lysosomal diameter after incorporation of Aβ compared to lysosomes in unstimulated cells (mean±s.e.m.). (e) For assessment of lysosomal integrity, microglia were incubated with the acidophilic/lysomotropic dye LysoTracker Red (red) and subsequently stimulated with fluorescent Hilyte-Aβ (green). Live cell imaging was performed using confocal microscopy. Phagocytosed Aβ showed a complete overlay with LysoTracker in small lysosomes (triangle), whereas enlarged Aβ-containing lysosomes lost LysoTracker fluorescence (arrows), indicating perturbation of lysosomal integrity. Scale bar=10 μm. (f) Flow cytometry analysis revealed that Aβ dose-dependently (1, 3 and 10 μM) induced loss of LysoTracker Green fluorescence in wild type microglia.

FIG. 13 shows the effects of lysosomal damage on cathepsin B release and the activation of the IL-1β pathway. (a) Microglial cells were stimulated with FITC-Aβ (green) for 1 h and 4 h, respectively, and processed for immunocytochemistry. Cathepsin B (red) was confined to small and round cellular compartments, consistent with lysosomal localization, under control conditions and after early Aβ stimulation (arrows). Lysosomal localization of cathepsin B was lost 4 h after Aβ phagocytosis, indicated by diffuse non-lysosomal cathepsin B-related fluorescence, suggesting release of cathepsin B by damaged lysosomes. Exclusion of cathepsin B from lysosomes was confirmed by staining with an antibody against LAMP-1 (cyan, inset). Cell nuclei were stained with Hoechst 33258 (blue). Scale bars=5 μm. (b) The cathepsin B inhibitor Ca-074-Me dose-dependently inhibited Aβ-induced IL-1β release from LPS-primed immortalized microglia. In contrast, no attenuation of the IL-1β surge was seen when cells were treated with pepstatin A or Z-FF-FMK, inhibitors of cathepsin D and L, respectively. The three cathepsin inhibitors in their highest concentration (10 μM) had no effect on IL-1β release induced by ATP. (c) A strong reduction of IL-1β release, as assessed by ELISA, was observed in primary macrophages from cathepsin B^{-/-} mice following stimulation with Aβ compared to wild type cells. In contrast, IL-1β release was equally strong in both cell types after stimulation with ATP or dAdT. (d) Microglial caspase-1 activation after Aβ stimulation was dose-dependently reduced by the cathepsin B inhibitor Ca-074-Me. Cathepsin B inhibition (20 μM) had no effect on caspase-1 activation induced by ATP. Caspase-1 activation was quantified using FLICA assay and flow cytometry (values represent mean fluorescence intensities).

FIG. 14 shows the role played by caspase-1 in the β-Amyloid-induced expression of pro-inflammatory and chemotactic factors in microglia. (a-b) Aβ-induced production of NO was strongly inhibited by the caspase-1 inhibitor z-YVAD-fmk in primed immortalized microglia (mean±s.e.m.) Immortalized cells from wild type and caspase-1^{-/-} mice failed to produce NO after stimulation Aβ for 24 h, but showed strong NO release after stimulation with the unspecific activator zymosan (10 μg/ml). (c-d) Likewise, production of TNF-α was inhibited by z-YVAD-fmk and did not occur in caspase-1^{-/-} cells. Supernatants were analyzed for TNF-α and Nitric Oxide by ELISA and Griess reaction 24 after stimulation, respectively. (e) Immortalized macrophages from wild type, ASC^{-/-}, NALP3^{-/-} or IPAF^{-/-} mice were stimulated with revAβ (10 μM), Aβ (10 μM), or LPS (100 ng/ml). NO production after stimulation with Aβ occurred only in wild type and IPAF^{-/-}, but was absent in ASC^{-/-} and NALP3^{-/-} cells. (f) Aβ-induced neuronal damage depends on microglial caspase-1. CAD mouse neuronal cells were co-cultured with microglia from wild type and caspase-1^{-/-} mice, respectively. Following stimulation with Aβ (10 μM, 72 h), cultures were fixed and stained with neuron-specific (TUJ-1, green) and microglia-specific (CD11b, red) antibodies. Aβ induced neuronal cell death in co-cultures from wild type mice, whereas neurotoxicity was strongly reduced in co-cultures with microglia from caspase-1^{-/-} mice. Aβ showed only small effects in neuronal mono-cultures. Cell nuclei were stained with Hoechst 33258 (blue). Scale bar=50 μm. (g) Real-time quantitative PCR of mRNA from primed immortalized microglia for chemokines CCL3, CCL4 and CXCL2 showed that caspase-1^{-/-} cells failed to upregulate these chemokines after stimulation with Aβ. RNA was extracted 6 and 24 h after stimulation.

FIG. 15 demonstrates the effects of Aβ and ATP treatment on the morphological and functional characteristics of immortalized mouse microglial cells. (a) Immortalized mouse microglial cells show 100% purity, as indicated by staining with the microglial antibody CD11b (green), and retain the typical morphology of primary microglia. These pure immortalized microglial cultures were derived from mixed primary glial cultures that contained microglia and astrocytes, as shown by staining with antibodies against CD11b (green) and the astrocytic marker GFAP (red). Note the morphological similarity between resting primary and immortalized microglia with typical fine processes (arrows). Scale bars=10 μm. Cell types in both cultures were quantified by flow cytometry, confirming that primary mixed cultures contained both microglia and astrocytes, whereas immortalized microglial cultures contained only microglia. (b) Immortalized and primary microglia express the same cell-surface markers. Cell-surface expression levels of CD14, CD45, CD11c, F4/80 and MHC II in primary microglia and immortalized microglial cells were analyzed by flow cytometry and compared. These data show that both microglial cultures used in this study showed consistent and highly comparable expression of microglia-typical cell surface markers. (c) Effects of Aβ stimulation on caspase-1 activation in primary microglia. Cells were either stimulated with Aβ (10 μM), revAβ (10 μM), ATP (1 mM), or left untreated. Caspase-1 activation (green) was measured using FLICA assay and confocal microscopy. Cell membranes were stained with choleratoxin subunit b (red). Scale bar=10 μm. (d) Effects of caspase-1 inhibition on IL-1β release in primary microglia. The caspase-1 inhibitor z-YVAD-fmk dose-dependently reduced IL-1β release after Aβ stimulation, as assessed by ELISA. As a positive control, IL-1β release was also inhibited by z-YVAD-fmk after stimulation with ATP (mean±s.e.m.).

FIG. 16 shows the effects on capase-1 activation, subsequent IL-1 signaling and the release of proinflammatory and neurotoxic factors by stimulation of microglia from IL-1 receptor−/− mice with Aβ. (a-b) Nitric oxide and TNF-α production following stimulation with Aβ were strongly reduced in primed microglia from IL-1 receptor^{-/-} mice. In contrast, no changes in the production of these factors were observed compared to wild type cells after stimulation with zymosan (mean±s.e.m.). (c) Aβ-induced IL-1β release was equally strong in IL-1 receptor^{-/-} microglia and wild type microglia (mean±s.e.m.).

FIG. 17 features a schematic model of NALP3 activation. Crystalline materials lead to lysosomal rupture and translocation of lysosomal content into the cytoplasm. Cathepsin B and potentially other proteases cleave a substrate leading to a NALP3 ligand. Assembly of NALP3 inflammasome proceeds under low potassium environment, leading to activation of caspase-1 and subsequent cleavage of pro-IL-β and pro-IL-18.

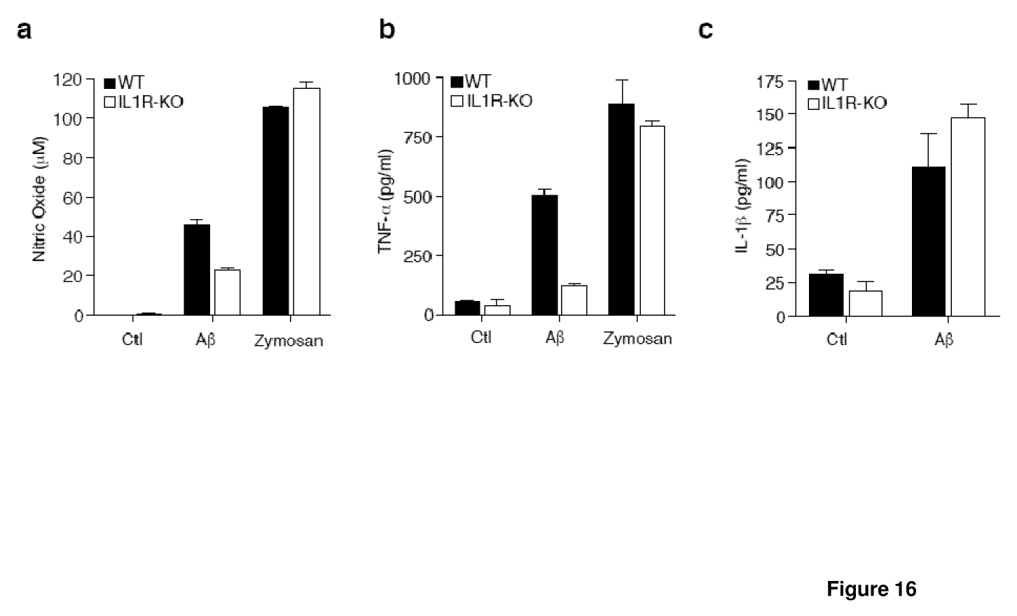

FIG. 18(a)-(b) shows the effects of crystal uptake on lysosomal structural integrity. (a) Flow cytometry of mouse macrophage cells incubated with increasing doses of DQ-ovalbumin in absence or presence of bafilomycin. (b) LPS-primed mouse macrophages were left untreated or were treated with bafilomycin and activated with ATP or silica crystals. Supernatants were assessed for IL-β by ELISA six hours after activation.

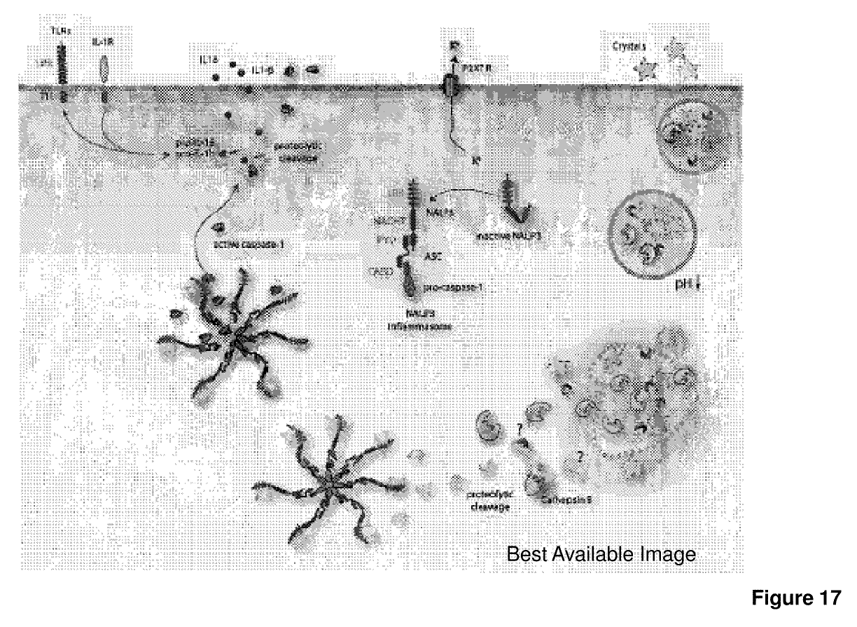

FIG. 18(c)-(d) shows the effects of cathepsin B on NALP3 activation by crystals. (c) LPS-primed wild-type mouse macrophages were incubated with increasing amounts of silica crystals, transfected dAdT or ATP in presence or absence of the cathepsin B inhibitor CA-074-Me. In (d) primary macrophages from wild-type or cathepsin B knock-out mice were stimulated with increasing amounts of silica crystals, transfected dAdT or ATP. IL-1β was determined in the supernatants after 6 h by ELISA. (e) Western Blot analysis of cathepsin B in supernatants of cells stimulated with crystals or controls show that cathepsin B maturation and release is independent of NALP3 and thus upstream of NALP3.

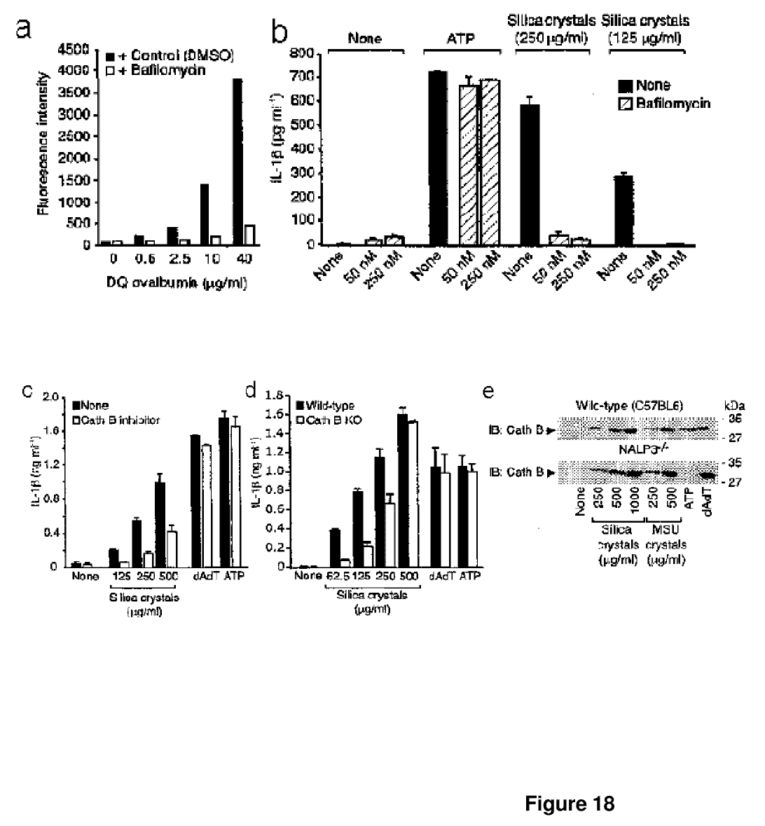

### DETAILED DESCRIPTION OF THE INVENTION

The present invention is based, at least in part, on the discovery that NALP3 inflammasome activation by pathogenic particles (e.g., crystals or fibers) is associated with the phagocytosis of the particles followed by lysosomal damage or leakage leading to the release of the pH-activated proteases into the cytosol. The present invention is further based on the discovery that NALP3 inflammasome activation and IL-1β production can be inhibited by lysosomal protease inhibitors (e.g., cathepsin inhibitors).

Accordingly, the present invention provides methods for treating inflammatory disorders induced by the disruption and/or acidification of endocytic vacuoles (e.g., lysosomes, phagosomes and/or endosomes) by administering an inhibitor of a pH-activated protease.

So that the invention may be more readily understood, certain terms are first defined.

I. Definitions

The articles “a” and ^{an} are used herein to refer to one or to more than one (i.e. to at least one) of the grammatical object of the article. By way of example, “an element” means one element or more than one element.

As used herein, the term “inflammatory disorder” includes diseases and disorders that are caused or primarily caused by inflammation, as well as diseases and disorders in which inflammation plays a role in the morbidity or symptoms of the disease or disorder, the propagation of the disease or disorder, the worsening of symptoms of a disease or disorder and/or the worsening of a patient's prognosis or survival time due to a disease or disorder.

As used herein, a “particulate-induced inflammatory disorder,” also referred to as an “inflammatory disorder induced by a particle”, is a disorder or disease in which an inflammatory response initiated by a pathogen particulate leads to the induction of the inflammatory disease or disorder, excessive inflammatory symptoms, host tissue damage, worsening of disease or disorder symptoms or a loss of tissue function.

The terms “particulate matter”, “particulate” and “particle” are used interchangeably herein and refer generally to one or more small aggregates of solid, precipitated and/or insolute material. Examples of particulate matter include, but are not limited to, crystals, protein or peptide aggregates, lipid aggregates, inorganic fibers, mineral aggregates, environmental pollutants, and cellular debris.

A “crystalline composition” or “crystallized composition” as used herein refers to composition containing molecules that form crystals at saturated concentrations, i.e., a concentration under which molecules in the composition undergo a chemical phase transition from the soluble (liquid) form to the solid (crystal) state. The composition can contain the molecules in a soluble form, which upon entry into the body, are capable of forming crystals upon deposition in the tissues. Alternatively, the crystalline composition can contain the molecules in crystalline form, e.g., as a suspension.

The term “pH-activated protease” or “acid-optimal protease” as used herein refers to a protease which is significantly or substantially more active at an acidic pH than at neutral or physiological pH ranges normally found within the cytosol of a cell, a tissue, an organism or a local environment. Exemplary proteases include those whose activity is triggered or increased in response to lysosomal acidification of the cytosol, such as cathepsin B and cathepsin L. The term “pH-activated protease inhibitor” or “acid-optimal protease inhibitor” as used herein refers to a substance (e.g., small organic molecule, antibody, peptide or nucleic acid) that prevents or reduces the enzymatic activity of a pH-activated protease.

The term “inflammasome(s)” is an art recognized term that refers to protein complex found in the cytosol which mediates the generation of proinflammatory cytokines, such as IL-33, IL-1β and IL-18. Inflammasomes contain a caspase enzyme, either caspase-1 or -5, that processes pro-cytokines into their active forms, a NOD-like receptor protein, such as NALP-3 (Martinon, F. et al. Sem. Immunopathology 29:213 (2007)), and ASC.

The term, “NALP3”, “cryopin”, “NLRP3”, “PYPAF1” and “CIAS1”, which may be used interchangeably herein, are art recognized terms that refers to a member of the Nod-like receptor family is expressed in both monocytes and macrophages (NM_004895, AF054176, AAR14737).

The adaptor protein “ASC” (“apoptosis-associated speck-like protein containing a caspase recruitment domain”) is a component of the inflammasome which binds to NALP3. ASC is required for proper inflammasome assembly (i.e., the recruitment of caspase-1), and thus for caspase-1 activation and the conversion of IL-1β by the inflammasome (Ferrero-Miliani, L. et al. Clin. Exp. Immunol. 147:227-235 (2006); Bouchier-Hayes, L. & Martin, S. EMBO Reports 3:616-621 (2002)). Alternative names for ASC in the art include PYCARD, CARDS, MGC10332, TMS1 and hASC.

The term “caspases” refers to a family of cysteine proteases that cleave proteins after aspartic acid residues. These proteins can be divided into two groups, the pro-apoptosis caspases and the proinflammatory caspases. The proinflammatory caspases include caspase-1 and caspase-5. These proteins generate inflammatory cytokines (e.g., IL-1β, IL-6, IL-18, IL-33) by converting the precursor versions of these cytokines into their mature, active forms.

“Caspase-1” as used herein is an art recognized term which refers to a member of the caspase family that is localized primarily in monocytes and serves to convert precursor IL-1β to the mature form (Black, R. A. et al., FEBS Lett., 247: 386-390 (1989); Kostura, M. J. et al., Proc. Natl. Acad. Sci. U.S.A., 86:5227-5231 (1989)). Enzymatically active caspase-1 is a heterodimer composed of two subunits, p20 and p10 (20 kDa and 10 kDa molecular weight, respectively). These subunits are derived from a 45 kDa proenzyme (p45) by way of a p30 form, through an activation mechanism that is autocatalytic. Thornberry, N. A. et al., Nature, 356, pp. 768-774 (1992). The caspase-1 proenzyme has been divided into several functional domains: a prodomain (p14), a p22/20 subunit, a polypeptide linker and a p10 subunit. (Thornberry et al., supra; Casano et al., Genomics, 20, pp. 474-481 (1994)). Caspase-1 is also known in the art as apoptosis-related cysteine peptidase, IL-1β converting enzyme (ICE) and IL-1β convertase.

The term ““Interleukin 1” (“IL-1”)” is an art recognized term that refers to a major pro-inflammatory and immunoregulatory protein that stimulates fibroblast differentiation and proliferation, the production of prostaglandins, collagenase and phospholipase by synovial cells and chondrocytes, basophil and eosinophil degranulation and neutrophil activation (Oppenheim, J. H. et al, Immunology Today, 7, pp. 45-56 (1986)).

The term “IL-1β” is an art recognized term that refers to an endogenous pyrogen which is a highly inflammatory cytokine. IL-1β is produced by the cleavage of a biologically inactive precursor, p IL-1β, by caspase-1 (Burns, K., Martinon, F. & Tschopp, J. Curr Opin Immunol 15, 26-30 (2003); (Martinon, F. et al Mol Cell 10, 417-26. (2002); Mariathasan, S. et al. Nature 430, 213-8 (2004).

“Patient” or “subject” as used herein includes living multicellular organisms, preferably mammals. The term “mammals” of the invention includes all vertebrates, e.g., such as nonhuman primates, sheep, dog, cat, horse, and cows. Examples of subjects include humans, dogs, cows, horses, kangaroos, pigs, sheep, goats, cats, mice, rabbits, rats, mice, hamsters and transgenic non-human animals. In preferred embodiments, the patient or subject is a human. In particular embodiments, the patient or subject is a human patient with an inflammatory disorder.

The term “treatment” or “treating” as used herein refers to either (1) the prevention of a disease (prophylaxis), or (2) the reduction or elimination of one or more symptoms of the disease of interest.

The terms “prevention”, “prevent” or “preventing” as used herein refers to inhibiting, averting or obviating the onset or progression of a disease (prophylaxis).

As used herein, an “effective amount” of a pH-activated protease inhibitor refers to an amount of protease inhibitor which, either alone or in combination with a pharmaceutically acceptable carrier, and upon single- or multiple-dose administration, prevents, reduces, alleviates and/or eliminates one or more symptoms of the inflammatory disorder.

As used herein, a “pharmaceutical composition” refers to a composition comprising a protease inhibitor, (e.g., a cathepsin B and or cathepsin L inhibitor), and a pharmaceutically acceptable carrier.

A “kit” is any manufacture (e.g., package or container) comprising at least one reagent, e.g., a pH-activated protease inhibitor), for use as inflammatory disorder in the methods of the invention. The kit can be promoted, distributed or sold as a unit for performing the methods of the invention.

II. Inflammatory Disorders

Inflammatory disorders which may be treated with protease inhibitors according to the methods of the invention include acute inflammations and chronic inflammations associated with vacuolar acidification and subsequent activation of the NALP3 inflammasome and II-1β secretion.

A. Particulate-mediated Inflammatory Disorders

Accordingly, in one aspect, the invention provides methods for treating inflammatory disorders associated with or induced by environmental or endogenous particulate agents (e.g., fibrous, crystalline or aggregate materials).

In some embodiments, the particulate-induced inflammatory disorder is associated with the tissue deposition of crystals. Nonlimiting examples of crystals that may induce inflammation include monosodium urate (MSU), basic calcium phosphate (BCP), calcium pyrophosphate dihydrate (CPPD), silica, hydroxyapatite, calcium oxalate, cholesterol, lipid liquid, other crystalline lipids, lithium heparin, and talc (magnesium silicate), starch crystals, cryoprotein crystals, lysophospholipase (Charcot-Leyden crystals), amyloid, ochronotic chards, hemoglobin, hematoidin, collagen fibrils, silicone, aluminum, cystine, xanthine, hypoxanthine crystals, and synthetic crystals. In certain, embodiments, the crystal is MSU, BCP, CPPD, silica or cholesterol.

Typically, crystals associated with induction of inflammatory responses are typically submicroscopic, having a length or diameter of about 0.1-300 μm, about 0.2-200 μm, about 0.5-100 μm, about 1-50 μm, about 1-40 μm, or about 2-20 μm and may vary in morphology, e.g. needle, rod or spherule, and size. The concentration of crystalline molecules may be elevated systemically, i.e, throughout the body by increased levels in the blood. Alternatively, the concentration of crystalline molecules may be elevated locally at the site of inflammation.

In some embodiments, the particulate-induced inflammatory disorder is associated with the uptake of environmental particulate matter. (e.g., by inhalation, ingestion, absorption, injection, etc.). Examples of environmental particulate matter include, but are not limited to, inorganic fibers (e.g., asbestos, cristobalite, man-made vitreous fibers), mineral particulates (e.g., inorganic dust, e.g., silica, asbestos, cristobalite, man-made vitreous fibers), byproducts of incomplete combustion (e.g., soot) as well as other environmental pollutants (e.g., diesel exhaust particles (DEPs), cigarette smoke extract (CSE)), and other dust (e.g., metal dusts, bacteria and animal dusts).

In other embodiments, the particulate-induced inflammatory disorder is associated with tissue deposition of endogenous biological materials released from damaged or dying cells or from necrotic cell debris that form particulate matter. Non-limiting examples of endogenous biological particles include aggregates of proteins (e.g., aggregated oxidized SOD1, aggregated a-Synuclein), other deposits of biological materials (e g , minimally modified LDL, aggregated lipoproteins, cholesterol, kidney stones), necrotic cells, apoptotic DNA, apoptotic cells and parts or pieces of dead cells.

Inflammatory disorders associated, at least in part, with exposure to endogenous or exogenous particulates that may be treated according to the methods of the invention include inflammatory disorders of the skeletal and muscular system, pulmonary disorders, cardiovascular disorders and neurological disorders.

For example, inflammatory disorders of the skeletal and muscular system associated with particulate-induced inflammation include, gout and pseudogout (e.g., uric acid crystals), and arthritis (e.g., necrotic cellular debris or apoptotic DNA)

Pulmonary inflammatory disorders include asbestosis, silicosis, fibrosis, ARDS Acute Respiratory Distress Syndrome (e.g., smoke inhalation), Chronic obstructive pulmonary disease (e.g., dead cells and debris arising from the destruction of lung parenchymal tissues and alveolar walls)

Cardiovascular inflammatory disorders include atherosclerosis (e.g., cholesterol crystals) and vasculitis (e.g., IgA dominant immune complex deposits), such as Henoch-Schönlein purpura cryoglobulinemic vasculitis, lupus vasculitis, serum sickness vasculitis and infection-induced immune complex vasculitis.

Neurological inflammatory disorders associated with deposition of particulate matter include Alzheimer's disease (e.g., aggregated amyloid), Parkinson's disease (e.g., aggregated a-Synuclein), Amyotrophic lateral sclerosis (ALS) (e.g., aggregated copper/zinc superoxide dismutase (SOD)), hereditary and sporadic prion disorders such as Creutzfeldt-Jacob disease (CJD), Gerstmann-Straiussler-Scheinker syndrome (GSS), and fatal familial insomnia (FFI) (e.g., aggregated, mutant and/or misfolded prion proteins and/or plaques comprising prion proteins such as fibrill protein aggregates).

B. NALP3-Mediated Inflammatory Disorders

In another aspect, the invention provides a method of treating inflammatory disorders associated with the activation of the NALP3 inflammasome and subsequent secretion of IL-β, by administering an inhibitor of a pH-activated protease (e.g., cathepsin B and/or cathepsin L).

In some embodiments, the methods of the invention may be used to treat autoinflammatory diseases, such as periodic fever syndromes. These diseases comprise a heterogeneous group of pathologies characterized by spontaneous periodic inflammation and fever in the absence of infectious or autoimmune causes and include familial Mediterranean fever (FMF); TNF receptor 1-associated periodic syndrome; Hyper-IgD syndrome; Periodic fevers with Aphthous stomatitis, Pharyngitis and Adentitis syndrome; pyogenic sterile arthritis, pyoderma gangrenosum and acne syndrome; Blau syndrome; and cryopyrin-associated periodic syndromes (e.g., familial cold autoinflammatory syndrome, Muckle-Wells syndrome and neonatal onset multisystem inflammatory disorder).

In some embodiments, the methods of the invention may be used to treat inflammation associated with autoimmune disorders including: diabetes mellitus, inflammatory bowel disease, arthritis (including rheumatoid arthritis, juvenile rheumatoid arthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, and interstitial lung fibrosis.

In some embodiments, the methods of the invention may be used to treat inflammation associated with neurological or neurodegenerative disorders including Parkinson's disease, prion disease, frontotemporal dementia, polyglutamine expansion diseases, spincocerebellar ataxia, spinal & bulbar muscular atrophy, spongiform encephalopathy, tauopathy, Huntington's disease, and dystonia.

In further embodiments, the methods of the invention may also be used to treat inflammation associated with hepatic disorders including alcoholic cirrhosis, al antitypsin deficiency, autoimmune cirrhosis, cryptogenic cirrhosis, fulminant hepatitis, hepatitis B and C, and steatohepatitis; biliary tract disorders including cystic fibrosis, primary biliary cirrhosis, sclerosing cholangitis and biliary obstruction.

In related embodiments, the methods of the invention may be used to treat inflammation resulting from a sterile inflammatory response, e.g., sterile tissue damage, hepatotoxicity, and drug induced heptatotoxicity. For example, in some embodiments the methods of the invention may be used to treat acetaminophen induced hepatotoxicity. It has been shown that DNA from apoptotic cells is linked to the activation of the NALP-3 inflammasome (see J Clin Invest. 2009 February; 119(2): 305-314 and J Clin Invest. 2009 February; 119(2): 246-349, which are incorporated herein by reference in their entirety). Accordingly, in some embodiments, the methods of the invention may be used to treat inflammation resulting from tissue necrosis, cell death, or apoptotic DNA, which has been shown to activate the NALP-3 inflammasome (J Clin Invest. 2009 February; 119(2): 305-314).

III. pH-Activated Protease Inhibitors

pH-activated proteases are a class of peptidases characterized by optimal activity in acidic environments. These include lysosomal proteases, such as lysosomal cathepsins and AEP. A number of cathepsins have been identified and sequenced from various sources (e.g, cathepsin B, C, F, H, L, K, 0, S, V, W, and Z). Cathepsin B, H, L and S are ubiquitously expressed lysosomal proteases.

Accordingly, in some embodiments, a pH-activated protease inhibited in the methods of the invention is a lysosomal protease. In one embodiment, the lysosomal protease is a cathepsin. In another embodiment, the cathepsin is selected from the group consisting of cathepsin B, H, L and S. In another embodiment, the cathepsin is cathepsin B. In another embodiment, the cathepsin is cathepsin L.

Inhibitors of pH-activated proteases, such as specific individual cathepsins or a subset of cathepsins, are known in the art. In some embodiments, the inhibitors which may be used in the methods of the invention are small molecules. Small molecule and peptide inhibitors of cathepsins have been described. For example, U.S. Pat. No. 6,458,760 describes small molecule or peptide inhibitors of cathepsins B and L, and U.S. Pat. No. 5,691,368 describes cysteine protease inhibitors that have specific activity in inhibiting cathepsins, both of which are hereby incorporated by reference in their entirety. Exemplary protease inhibitors that inhibit cathepsin B include CA074Me and E64d (Hook, V. et al. J. Biol. Chem. 283:7745-53 (2008)).

In other embodiments, the protease inhibitors may be a nucleic acid silencing agents (e.g., RNA or DNA silencing agents). In certain embodiments, the nucleic acid silencing agents are silencing agents having sufficient complementarity to a target RNA (e.g., an mRNA comprising sequences from the gene of a pH-activated protease (e.g., cathepsin B or cathepsin L) to mediate gene silencing of the target RNA. In certain embodiments, gene silencing is such that the target gene level is reduced or decreased by at least 30%. In other embodiments, gene silencing is such that target gene levels are reduced or decreased by at least at least 40% (e.g., at least 40%, at least 50%, at least 60%, at least 70%, at least 80%, at least 90%, or more). The nucleic acid silencing agents may be single or double stranded. For example, the nucleic acid silencing agents may be RNA silencing agents comprising or consisting of an antisense RNA strand (or portions thereof) comprising a sequence with sufficient complementarity to the target mRNA to silence expression of a target mRNA via an RNA silencing mechanism (e.g., RNA interference (RNAi) or translational repression). In other embodiments, the nucleic acid silencing agent may be DNA silencing agents comprising or consisting of an antisense strand comprising a sequence with sufficient complementarity to the target RNA to silence expression via an antisense mechanism (e.g., via RNase H mediated cleavage). In yet other embodiments, the nucleic acid silencing agents may be inhibitors of RNA silencing. For example, the nucleic acid silencing agents may comprise or consist of an antisense strand comprising a sequence with sufficient complementarity to a small, non-coding RNA (e.g., a miRNA, pre-miRNA, pri-miRNA, rasi-RNA, smRNA or piRNA) to inhibit RNA silencing by the RNA (e.g., so-called “antagomiRs”).

Additional protease inhibitors may be identified using screening methods that are well known in the art (e.g., Leist et al. J. Immunol. 154:1307-1316, 1995; Guicciardi et al. J. Clin. Invest. 106:1127-1137, 2000; European Patent No. 1172443 or WO/2007/017293). Accordingly, methods provided herein can also be used to identify an agent useful for reducing inflammation associated with pH-activated protease activity. In one embodiment, a method useful for identifying an agent useful for cell injury involves contacting a cell containing an activated protease with a test agent and assaying for an indicator protease activity, whereby an agent is identified based on its ability to reduce protease activity in comparison to a suitable control. A “suitable control” is any control or standard familiar to one of ordinary skill in the art useful for comparison purposes. For example, suitable controls can be an appropriate solvent or dispersion medium, e.g., containing, for example, water, ethanol, polyol (e.g., glycerol, propylene glycol and liquid polyethylene glycol and the like) and suitable mixtures thereof used for dilution and/or delivery of the potential protease inhibitor to the cells or subject. Suitable controls can be, for example, cell culture medium, PBS, saline and the like.

IV. Pharmaceutical Compositions and Methods of Administration

The present invention also relates to the use of an inhibitor of a pH-activated protease (e.g., cathepsins such as B and or L) for the preparation of a medicament used in the treatment of an inflammatory disorder. For example, an agent that decreases the activity of a pH-activated protease (e.g., cathepsin B and/or L) can be incorporated into pharmaceutical compositions suitable for administration. Such compositions typically comprise the agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. A pharmaceutical composition of the invention is formulated to be compatible with its intended route of administration.

The protease inhibitors used in the methods of the invention may be administered systemically, i. e, throughout the body by increased levels in the blood, or may be administered locally at the site of inflammation. Accordingly, embodiments of the invention feature protease inhibitors that are administered using standard administration techniques, preferably peripherally by injection or infusion, intravenous, intraperitoneal, intramuscular or subcutaneous, but also by other routes such as pulmonary, intranasal, buccal, sublingual, transdermal, oral, or suppository administration.

The term “administering” includes any method of delivery of a pharmaceutical composition or agent into a subject's system or to a particular region in or on a subject. The phrases “systemic administration,” “administered systemically”, “peripheral administration”, and “administered peripherally” as used herein mean the administration of a compound, drug or other material other than directly into the central nervous system, such that it enters the subject's system and, thus, is subject to metabolism and other like processes, for example, subcutaneous administration. “Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

In most embodiments, the agent (e.g., an inhibitor of a pH-activated protease) will be delivered in an amount sufficient to deliver to a subject a therapeutically effective amount of the agent as part of a prophylactic or therapeutic treatment. The desired concentration of the agent will depend on absorption, inactivation, and excretion rates of the agent as well as the delivery rate of the agent. It is to be noted that dosage values may also vary with the severity of the condition to be alleviated. It is to be further understood that for any particular subject, specific dosage regimens should be adjusted over time according to the individual need and the professional judgment of the person administering or supervising the administration of the agent. Typically, dosing will be determined using techniques known to one skilled in the art. The selected dosage level will depend upon a variety of factors including the activity of the particular agent, the route of administration, the time of administration, the rate of excretion or metabolism of the agent, the duration of the treatment, other drugs, compounds and/or materials used in combination with the agent, the age, sex, weight, condition, general health and prior medical history of the patient being treated, and like factors well known in the medical arts.

Dosage may be based on the amount of the composition per kg body weight of the patient. Other amounts will be known to those of skill in the art and readily determined. Alternatively, the dosage of the subject invention may be determined by reference to the plasma concentrations of the composition. For example, the maximum plasma concentration (Cmax) and the area under the plasma concentration-time curve from time 0 to infinity (AUC (0-4)) may be used. Dosages for the present invention include those that produce the above values for Cmax and AUC (0-4) and other dosages resulting in larger or smaller values for those parameters.

A physician or veterinarian having ordinary skill in the art can readily determine and prescribe the effective amount of the agent required. For example, the physician or veterinarian could start doses of the agent at levels lower than that required in order to achieve the desired therapeutic effect and gradually increase the dosage until the desired effect is achieved.

In general, a suitable dose of an agent will be that amount which is the lowest dose effective to produce a therapeutic effect. Such an effective dose will generally depend upon the factors described above. The amount of protease inhibitor administered by local inoculation will typically range from about 10 ng to about 10 mg per site, preferably about 10 μg to about 1 mg per site. For systemic administration, inoculations of protease inhibitor may contain about 1 mg/kg to about 1000 mg/kg of body weight.

In the method according to the invention, inhibitors of a pH-activated protease (e.g., cathepsin B and or L) may be used combined with other therapeutic agents, such as known anti-inflammatory compounds and/or in combination with inhibitors of additional components of the NALP3 inflammasome pathway. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly.

For example, in some embodiments, anti-inflammatory compounds which may be used in the methods of the invention include and any anti-inflammatory compound listed in the pharmacopea. Exemplary compounds include, but are not limited to corticoids, such as prednisone, betamethasone, dexamethasone, methylprednisolone, prednisolone, cortivazol, hydrocortisone, triamcinolone, and non steroids such as indometacine, sulindac, tiaprofenic acid, alminoprofene, diclofenac, etodolac, flurbiprofene, ibuprofene, ketoprofene, nabumetone, naproxene, meloxicam, piroxicam, tenoxicam, celecoxib, refecoxib. “Nonsteroidal anti-inflammatory drugs” or NSAIDS, inhibit the metabolism of arachidonic acid to proinflammatory prostaglandins via cyclooxygenase (COX)-1 and COX-2. Nonlimiting examples of NSAIDs include: aspirin, ibuprofen, naproxen, diclofenac, etodolac, fenoporfen, flubiprofen, indomethacin, ketoprofen, ketorolac, meloxicam, nabumetone, oxaprozin, piroxicam, sulindac, tolmetin, diflunisal, meclofenamate and phenylbutazone.

In some embodiments, the protease inhibitors may be administered with IL-1 inhibitors. Interleukin-1 inhibitors are known in the art, for example, in WO 89/11540 and U.S. Pat. No. 6,417,202. In one embodiment, the inhibitor of IL-1 is an IL-1 receptor type I (IL-1R1) antagonist, natural or synthetic, e.g., IL-1Ra also known as anakira, marketed under the name Kineret™. In another embodiment, the IL-1 inhibitor is an antibody which inhibits the activity of IL1α or IL1β. Such antibodies are known in the art, (see for example WO 03/073982, enclosed herein by reference). In another embodiment, the inhibitor is an IL1β, e.g., diacerein and rhein.

In other embodiments of the invention for the treatment of gout and pseudogout may comprise the administration of known anti-gout compounds and compositions, such as colchicines and compositions for preventing the accumulation of uric acid. Examples of agents that can be used to reduce the concentration of uric acid in a subject include, but are not limited to, agents that increase excretion of uric acid (e.g. uricosuric agents), agents that inhibit uric acid synthesis and agents capable of degrading uric acid. Examples of agents useful for reducing uric acid concentration in a subject include, but are not limited to uricase, allopurinol, probenicid, sulfinpyrazone, benzbromarone, zoxazolamine, diflunisal, aspirin and tienilic acid, E5050, FK366, CGS12970, Ambroxol and AA193 (5 chloro-7,8-Dihydro-3-phenyl furol (2,3G)-1,2-bensizoxazole). Anti- MSU crystal antibodies are also known to those skilled in the art, e.g., see Kam M., Perl-Treves D., Sfez R., Addadi L. (1994) “Specificity in the recognition of crystals by antibodies” Journal of Molecular Recognition 7(4):257-64.

In some embodiments, the protease inhibitors may be administered in combination with HSP90 inhibitors. HSP90 inhibitors known in the art include, but are not limited to those disclosed in U.S. Pat. No. 4,261,989, US 2004-0235813, WO 02/36574, WO 02/079167, WO 03/02671, WO 2005/095347, WO 2006/095783, WO 2006/092202, WO 2006/090094, WO 2006/087077, WO 2006/084030, WO 2005/028434, WO 2004/072051, WO 2006/079789, US 2006-0167070, WO 2006/075095, US 2006-0148817, WO 2006/057396, WO 2006/055760, WO 02/069900, WO 2006/052795, WO 2006/050373, WO 2006/051808, WO 2006/039977, US 2006-0073151, EP 1 642 880, EP 1 631 267, EP 1 628 667, US 2006-0035837, WO 2006/008503, WO 2006/010595, WO 2006/010594, WO 2006/003384, WO 2005/115431, EP 1 620 090, WO 2005/061461, WO 2005/063222, US 2005-0049263, WO 2004/050087, WO 2004/024142, WO 2004/024141, WO 03/067262, WO 03/055860, WO 03/041643, WO 03/037860. In certain embodiments, the HSP90 inhibitor used in the methods of the invention may be geldanamycin, or derivatives thereof such as 17-AAG (17-(Allylamino)-17-demethoxygeldanamycin) or 17-DMAG (17-(Dimethylaminoethylamino)-17-demethoxygeldanamycin) and their pharmaceutically acceptable salts.

In other embodiments, agents capable of inhibiting inflammation are any agents that would modulate, e.g., downmodulate, e.g., inhibit the ability of a cathepsin molecule (e.g., cathepsin B) to activate NALP3 -ASC-capsase-1 complex formation and/or inflammasome activity.

The invention is further illustrated by the following examples, which should not be construed as further limiting. The contents of all references, pending patent applications and published patents, cited throughout this application are hereby expressly incorporated by reference.

### EXAMPLES

Reference is made to standard textbooks of molecular biology that contain definitions and methods and means for carrying out basic techniques, encompassed by the present invention. See, for example, Sambrook et al., Molecular Cloning: A Laboratory Manual, Third Edition, Cold Spring Harbor Laboratory Press, New York (2001), Current Protocols in Molecular Biology, Ausebel et al (eds.), John Wiley & Sons, New York (2001) and the various references cited therein.

The following Examples 1 to 8 describe experiments investigating the ability of silica to activate the NALP-3 inflammasome.

Material and Methods for Examples 1 to 8:

Mice:

NALP3¹³-, ASC¹³- and IPAF³⁶-deficient mice were obtained from Millennium Pharmaceuticals (Cambridge, Mass.). C57BL/6 mice, 129/Sv mice, C57/B16×129 F1 mice, IL-1R^{-/-} and gp9lphox^{-/-} were purchased from Jackson Laboratories (Bar Harbor, Me.). MyD88^{-/-}-TRIF^{-/-}mice were generated from MyD88^{-/-} and TRIF^{-/-} mice, obtained from Shizuo Akira (Kyoto, Japan). Seven to nine week-old animals were used in all experiments. All mouse strains were bred and maintained under specific pathogen-free conditions.

Reagents:

Acridine orange, ATP, bafilomycin Al, cytochalasin D, LPS, PMA, poly(deoxyadenylic-thymidylic) acid sodium salt (dAdT), sucrose and zymosan were purchased from Sigma-Aldrich (St. Louis, Mo.). CA-074-Me and PEG 1000 were purchased from Calbiochem (Gibbstown, N.J.). DQ-ovalbumin, A647-conjugated dextran, A647-conjugated choleratoxin B, lysosensor green and Hoechst stain were obtained from Molecular Probes, Invitrogen (Carlsbad, Calif.). Alum (Imject Alum Adjuvant, mixture of aluminum hydroxide and magnesium hydroxide) was purchased from Pierce (Rockford, Ill.). Leu-Leu-OMe-HCl was purchased from Chem-Impex International (Wood Dale, Ill.). Uricase (Elitek™) was purchased from Sanofi-Aventis (Bridgewater, N.J.). Silica crystals (MIN-U-SIL-15) were obtained from the U.S. Silica Company (Berkeley Springs, W. Va.). Throughout the study, a polydispersed preparation of silica crystals of up to 15 μm was used. MSU crystals were prepared as previously described (Schiltz, C. et al. Arthritis and rheumatism 46:1643-1650 (2002)).

In vivo silica model:

Mice were exposed to 40 μl aqueous suspensions of 200 μg of silica crystals (MIN-U-SIL-15) in PBS or 50 μg crude zymosan by direct orotracheal instillation. Control mice received PBS Animals were sacrificed 16-18 hours after instillation and a bronchoalveolar lavage (BAL) was carried out by repeatedly instillating and withdrawing 1 ml of 1% BSA/PBS solution three consecutive times. Recovered BAL fluid was pelleted by centrifugation and counted. Subsequently, cells were stained for surface markers and neutrophils were identified as double positive cells for MCA771B (Serotec, Raleigh N.C.) and Ly6G (BD Biosciences) using flow cytometry. For alum experiments, mice were injected i.p. with 100 μg of a mixture of aluminum hydroxide and magnesium hydroxide (Pierce, Rockford, Ill.) in 200 μl PBS. 16-18 h after challenge, animals were euthanized and their peritoneal cavities were washed with 6 ml PBS containing 3 mM EDTA and 10 U/ml heparin. Total numbers of peritoneal exudate cells were counted by a hematocytometer, and the numbers of neutrophils were evaluated as described above.

Cell Isolation and Culture:

Bone marrow derived macrophages were generated as described (Severa, M et al. Journal of Biological Chemistry 281:26188-26195 (2006)). Human PBMCs were isolated by from whole blood of healthy volunteers by density gradient centrifugation. Lysis of red blood cells was performed using red blood cell lysis buffer (Sigma). Experiments in PBMCs and macrophages were carried out at a cell density of 2×10⁶ cells/ml. All primary cells and cell lines except THP-1 cells were cultured in DMEM supplemented with L-glutamine, ciprofloxacin (Cellgro, Manassas, Va.) and 10% fetal calf serum (Hyclone, Logan, Utah). THP-1 cells were cultured in RPMI supplemented with 10% fetal calf serum (Hyclone), L-glutamine, sodium pyruvate (Cellgro), and ciprofloxacin. One day prior to stimulation, THP-1 cells were differentiated using 0.5 μM PMA for three hours, washed three times and plated for stimulation. All experiments that were performed for Western blot analysis were carried out in serum free DMEM medium. ATP stimulations were carried out at 5 mM one hour prior to harvesting supernatants.

Immortalized Macrophage Cell Lines:

Immortalized macrophage cell lines were generated using the previously described J2 recombinant retrovirus (carrying v-myc and v-raf/mil oncogenes) (Roberson, S. M. & Walker, W. S. Cellular immunology 116:341-351 (1988)). Briefly, primary bone marrow cells were incubated in L929 conditioned medium for 3-4 days to induce macrophage differentiation. Subsequently, cells were infected with J2 recombinant retrovirus. Cells were maintained in culture for 3-6 months slowly weaning off the percentage of L929 supernatant until cells were growing in the absence of conditioned medium.

Macrophage phenotype was verified by surface marker expression for CD11b and F480 as well as a range of functional parameters, including responsiveness to TLR ligands and bacterial uptake. Macrophage cell lines from wild-type (C57BL6), NALP3- and ASC-deficient mice were generated and are referred to as B6-MCLs, NALP3 -MCLs and ASC-MCLs.

Flow Cytometry Analysis:

For evaluation of lysosomal rupture, cells were incubated with 1 μg/m1 acridine orange for 15 min, washed three times and subsequently stimulated as indicated. Lysosomal rupture can be assessed by loss of emission at 600-650 nm using flow cytometry. All flow cytometry experiments were performed on an LSRII cytometer (BD Biosciences). Data were acquired by DIVA (BD Biosciences) and analyzed by FlowJo software (Tree Star Inc., Ashland, Oreg.).

Confocal Microscopy:

Confocal reflection microscopy was combined with fluorescence microscopy on a Leica SP2 AOBS confocal laser scanning microscope. Reflection was captured by placing the detector channel directly over the wavelength of the selected laser channel for reflection light capture and the AOBS was set to allow 5-15% of laser light into the collection channel. Fluorescence was simultaneously captured by standard confocal imaging techniques.

ELISA:

Cell culture supernatants were assayed for IL-1β using ELISA kits from BD Biosciences (Franklin Lakes, NJ) according to the manufacturer's instructions. To measure intracellular IL-1β, cells were washed and subjected to three freeze thaw cycles in assay diluent.

Lysosomal Rupturing in THP-1 Cells:

PMA-differentiated THP-1 cells were incubated with hypertonic DMEM medium containing 10% polyethylene glycol (PEG) 1000, 1.4 M sucrose, 20 mM Hepes (pH 7.2) and 5% FCS for 10 mM at 37° C. Cells were subsequently washed and incubated in hypotonic DMEM medium [DMEM: H₂O (3:2)] for 2 min to induce lysosomal rupturing. Cells were then incubated in serum free DMEM for additional 4 hours.

Staining of Cathepsin B and Caspase-1:

After treatment as indicated, cells were incubated with the fluorescent cathepsin B substrate Magic Red (cresyl violet, bisubstituted via amide linkage to the dipeptide arginine-arginine) together with the FLICA caspase-1 green fluorescent peptide (FAM-YVAD-FMK) for 30 min at 37 C per the recommendations of the manufacturer (Immunochemistry Technologies, Bloomington, Minn.). After three washes in PBS, cells were visualized by confocal microscopy.

Western Blot Analysis:

Cell culture supernatants were precipitated by adding an equal volume of methanol and 0.25 volumes of chloroform, vortexed and centrifuged at 20.000×g for 10 min. The upper phase was discarded and 500 μl of methanol was added to the interphase. This mixture was centrifuged at 20.000×g for 10 min and the protein pellet dried at 55° C., resuspended in Laemmli buffer and boiled at 99° C. for 5 min. Samples were separated by SDS-PAGE (15%) and transferred onto nitrocellulose membranes. As indicated, blots were incubated with rabbit polyclonal antibody to anti murine caspase-1 p10 (sc-514, Santa Cruz Biotechnology, Santa Cruz, Calif.), rabbit polyclonal anti human caspase-1 p10 (sc-515, Santa Cruz Biotechnology), rabbit polyclonal anti human cleaved IL-1β (Asp116) (Cell Signaling, Boston, Mass.) or rabbit polyclonal anti murine cathepsin B (R&D Systems, Minneapolis, Minn.).

### Example 1

### Silica Induces Release of Mature IL-1β and Activated Caspase-1 in Human PBMCs

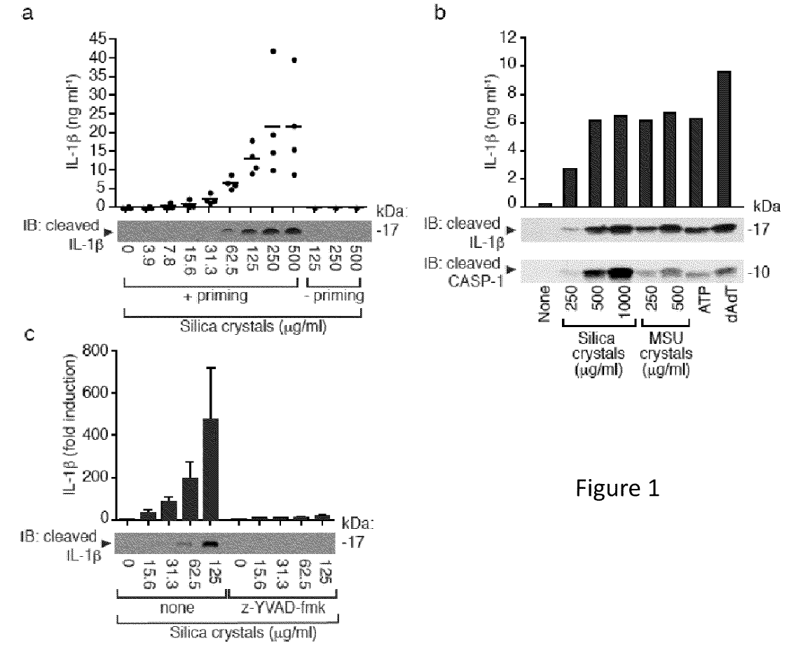

Human PBMCs from several donors were incubated with baked LPS-free silica crystals. Pro-IL-1β is not constitutively expressed and requires transcriptional induction in response to e.g. a TLR stimulus. While silica crystals did not induce IL-1β cleavage and release in human PBMCs by themselves, LPS-primed PBMCs strongly responded to the addition of silica crystals in a dose-dependent manner (FIG. 1a). Silica crystal-mediated activation of human PBMCs was as potent as other known activators of the NALP3 inflammasome, such as MSU crystals, ATP or the NALP3 -independent stimulus transfected double-stranded DNA (dAdT)⁷ (FIG. 1b). IL-1β production as measured by ELISA correlated strongly with the detection of cleaved IL-1β and cleaved caspase-1 as assessed by Western blotting, indicating that activated cells release mature IL-1β Inhibition of caspase-1 by the specific peptide inhibitor z-YVAD almost completely abolished the IL-1β response in response to silica crystal treatment (FIG. 1c). These data suggest that silica crystals activate IL-1β in a caspase-1 dependent manner in human immune cells.

### Example 2

### Silica-mediated Neutrophil Influx in a Model of Acute Lung Inflammation is Mediated by IL-1

After exposed people inhale silica crystal dust, lung-resident immune cells subsequently engulf the crystal material by phagocytosis and induce an inflammatory response. Large doses of silica dust lead to the clinical syndrome of acute silicosis, characterized by the rapid influx of immune cells into the exposed area and massive production of chemokines and proinflammatory cytokines, including IL-1β2. To investigate the in vivo relevance of the IL-1 response in silica-induced lung inflammation, we transorally instilled silica crystals into wildtype and IL-1 receptor (IL-1R)-deficient mice and then monitored the mice for acute inflammation after 16-18 h. We counted the cells in bronchoalveolar lavage fluid and assessed their phenotype by flow cytometry. We detected much more infiltration of neutrophils in lungs of wild-type mice exposed to silica crystals than in those of mice treated with the carrier fluid; this did not occur in IL-1R-deficient mice exposed to silica crystal (FIG. 1d). Other cell types remained mostly unchanged (data not shown).

We noted almost complete abrogation of silica-induced influx of neutrophils in mice doubly deficient in the adaptor molecules MyD88 (A003535) and TRIF, whereas absence of these molecules had little or no effect on zymosan-induced inflammation (FIG. 1e). Commercial zymosan preparations are known to activate signaling pathways dependent on Toll-like receptor 2 and dectin 1 ^{8.9}, which could explain the somewhat lower zymosan-induced neutrophil recruitment. These results collectively indicate that silica crystals induce IL-1 release and that the IL-1 signal-transduction molecules IL-1R and MyD88 are critical in the development of acute inflammation in vivo after exposure to silica crystal.

### Example 3

### Silica Crystals Activate the NALP3 Inflammasome

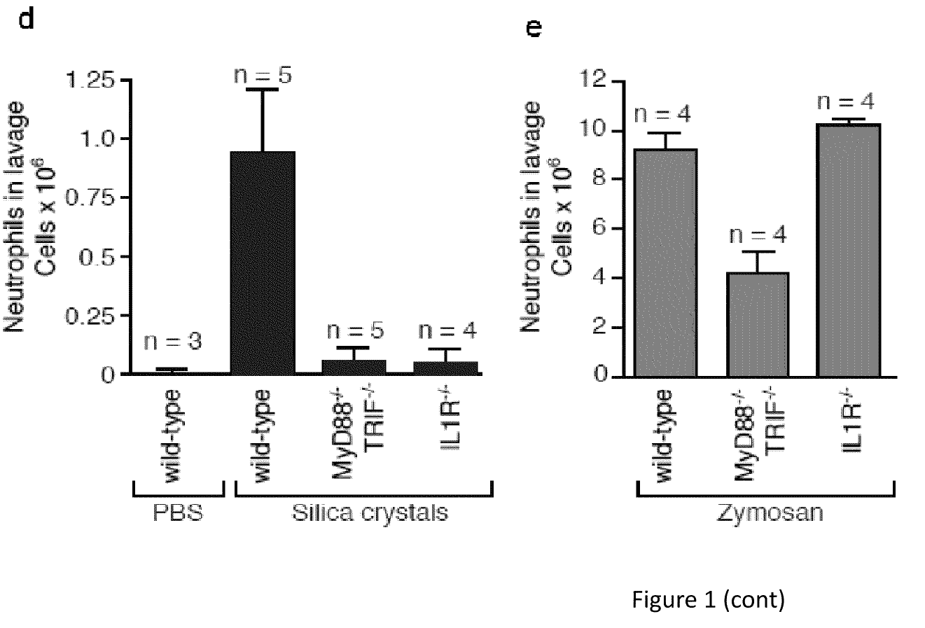

To investigate whether silica crystals can activate the NALP3 inflammasome, experiments were performed with macrophages from mice deficient in NALP3 or the ‘downstream’ adaptor molecule ASC. Similar to their response to known inflammasome activators such as MSU crystals, ATP or transfected poly(dA:dT), macrophages from wild-type mice produced large amounts of IL-1β after exposure to silica crystals (FIG. 2a, left). In contrast, macrophages lacking NALP3 or ASC failed to release cleaved IL-1β in response to silica crystals (FIG. 2a), which indicated a requirement for NALP3 and ASC in the processing of IL-1β after exposure to silica crystals. Consistent with published Q5 reports 3,7,10,11, responses to MSU crystals and ATP were dependent on both NALP3 and ASC, whereas the response to transfected poly(dA:dT) was ASC dependent yet NALP3 independent (FIG. 2a, middle and left).

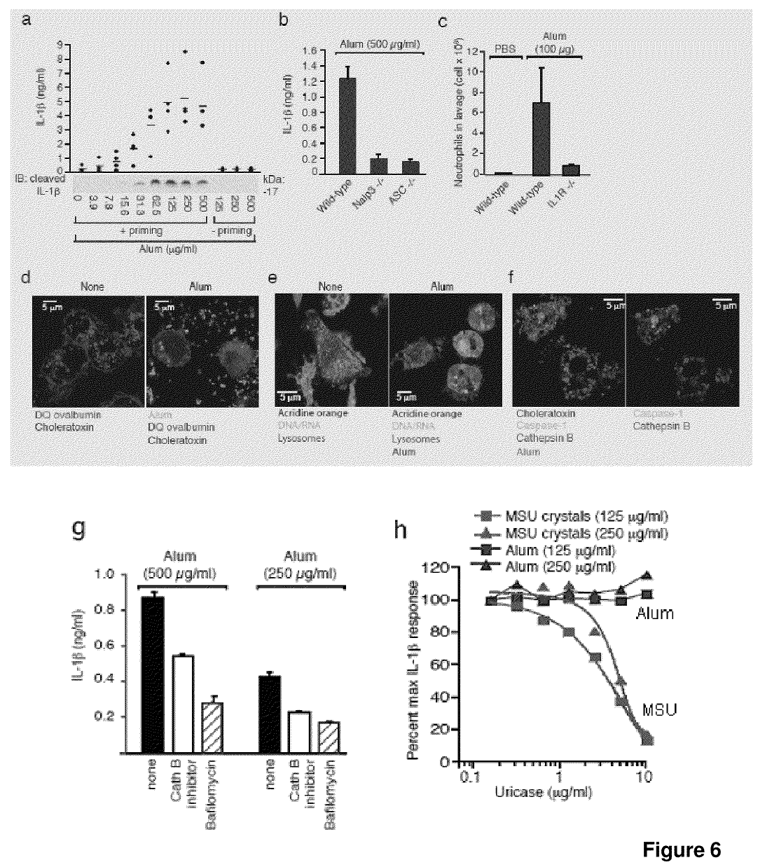

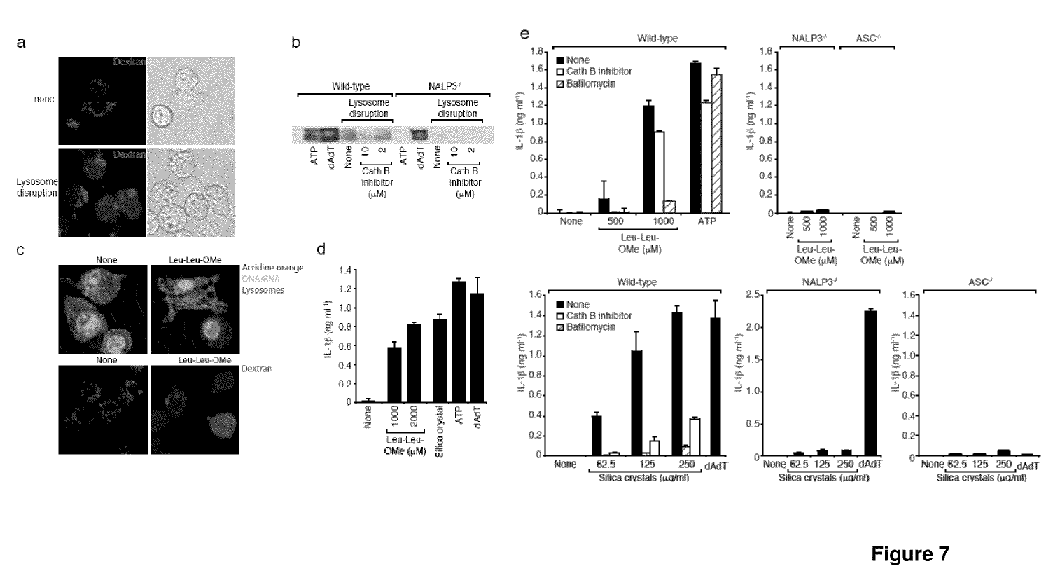

As IL-1β processing and release could both be influenced by activation by silica crystals, the cleavage of procaspase-1 into active caspase-1 were next examined, and the results indicate that, like the release of IL-1β, caspase-1 cleavage was induced in a dose dependent way after treatment with silica crystals, MSU, ATP or poly(dA:dT) (FIG. 2b). This response was completely dependent on ASC for all stimuli, as ASC-deficient macrophages failed to trigger caspase-1 cleavage in response to any of these ligands. Consistent with the IL-1β release, caspase-1 cleavage in response to poly(dA:dT) was independent of NALP3. A recent report has suggested uric acid released from cells is a trigger for IL-1β production after stimulation of cells with other crystal preparations¹². We assessed whether uricase influenced the IL-1β response to MSU or silica crystals and found that the response to silica was unimpaired after incubation with uricase (FIG. 2c). We also generated immortalized macrophage cell lines from wild-type, NALP3- and ASC-deficient mice and examined their response to inflammasome ligands Immortalized macrophages had responses qualitatively similar to those of freshly isolated bone marrow-derived macrophages (FIG. 8a). These results collectively suggest that silica crystals activate the NALP3-ASC complex, leading to the activation of caspase-1 and subsequent cleavage of pro-IL-1β into mature, secreted IL-1β.

### Example 4

### Crystal Uptake is Required for NALP3 Inflammasome Activation

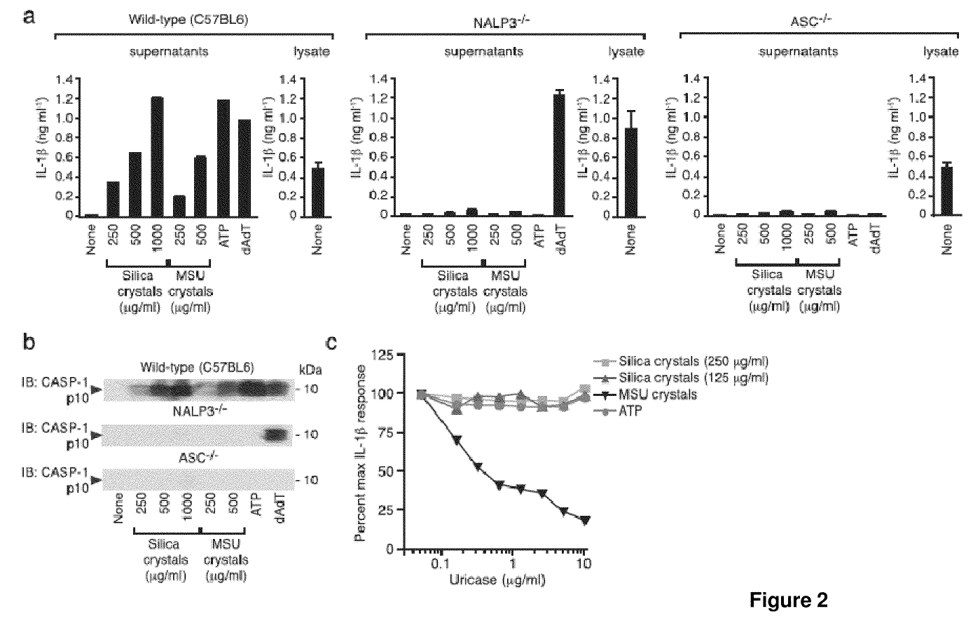

To delineate the ‘upstream’ mechanisms involved in silica crystal-induced activation of the NALP3 inflammasome, we first determined if uptake of crystalline inflammasome activators influenced cell activation. We pretreated human PBMCs with cytochalasin D, a well characterized inhibitor of phagocytosis that impairs actin filament assembly, and then stimulated the cells with MSU or silica crystals as well as with two noncrystalline NALP3 activators, the imidazoquinoline derivative R-848 and ATP13. Cytochalasin D completely abrogated IL-1β release after treatment with MSU or silica crystals, whereas the response to R-848 or ATP was unaffected (FIG. 3a).

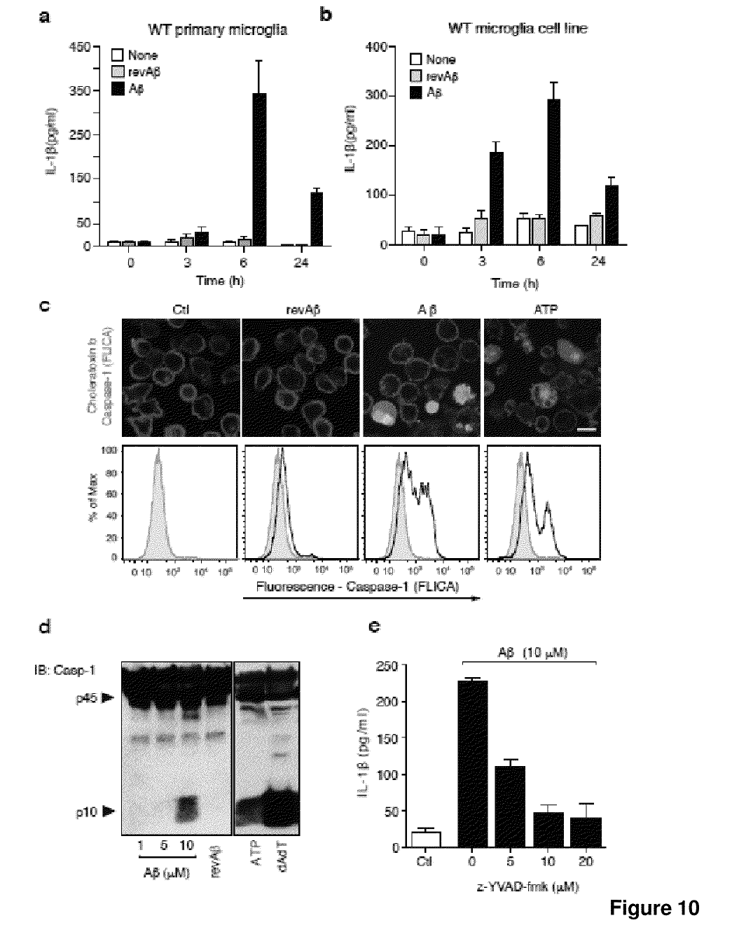

We obtained similar results with mouse macrophages (FIG. 10a).

FIG. 10a shows the effects of cytochalasin D (a crystal uptake inhibitor) on the crystal-mediated inflammasome activation in LPS-primed B6-MCLs which were treated with cytochalasin D for 30 min and subsequently stimulated with ATP, MSU crystals (250 μg/ml) and silica crystals (500 μg/m1) (release of IL-1β was assessed by ELISA). The results show that cytochalasin D abrogated IL-1β release after treatment with MSU or silica crystals, whereas the response to ATP was unaffected. These results indicate crystal binding and uptake by phagocytosis are prerequisites for activation of the cytosolic NALP3 inflammasome.

To visualize the uptake of crystalline material in living cells, we devised a method to image crystals simultaneously with cellular components stained with fluorescent dyes (FIG. 8b). We used this method to study the uptake of crystals into mouse macrophages. After incubating cells with silica crystals for 30 min, we stained the cell surface with the membrane-binding reagent fluorescent cholera toxin B-subunit in the presence or absence of cytochalasin D. Macrophages rapidly engulfed crystals into intracellular compartments by phagocytosis, whereas cells treated with cytochalasin D failed to engulf silica crystals (FIG. 3b) or MSU crystals (FIG. 3f) by phagocytosis. The median length of silica Q6 crystals engulfed by phagocytosis was 1.65 μm (FIG. 3c).

It is well known that phagocytosis of particulate matter in macrophages results in the generation of reactive oxygen species. Silica crystals trigger the production of reactive oxygen species in macrophages, events reported to be linked to disease pathology in silica-induced inflammation¹⁴. On the basis of those reports and our own data emphasizing the importance of phagocytosis in silica crystal-induced NALP3 activation, we hypothesized that the phagocyte reactive oxygen species system might be involved in this response. To address the involvement of this pathway directly, we examined NALP3 inflammasome responses in mice lacking gp91phox, the 91-kilodalton subunit of the phagosomal NADPH oxidase cytochrome b. Mice deficient in this subunit lack phagocyte superoxide production and are reported to be hypersusceptible to various pathogens¹⁵. However, macrophages from these mice responded normally to silica crystals, MSU, ATP and poly(dA:dT) (FIG. 3d,e). These results indicate that the phagosomal respiratory-burst oxidase system is not essential for activation of the NALP3 inflammasome by these stimuli.

### Example 5

### Phagocytosis of Crystals Leads to Lysosomal Destabilization

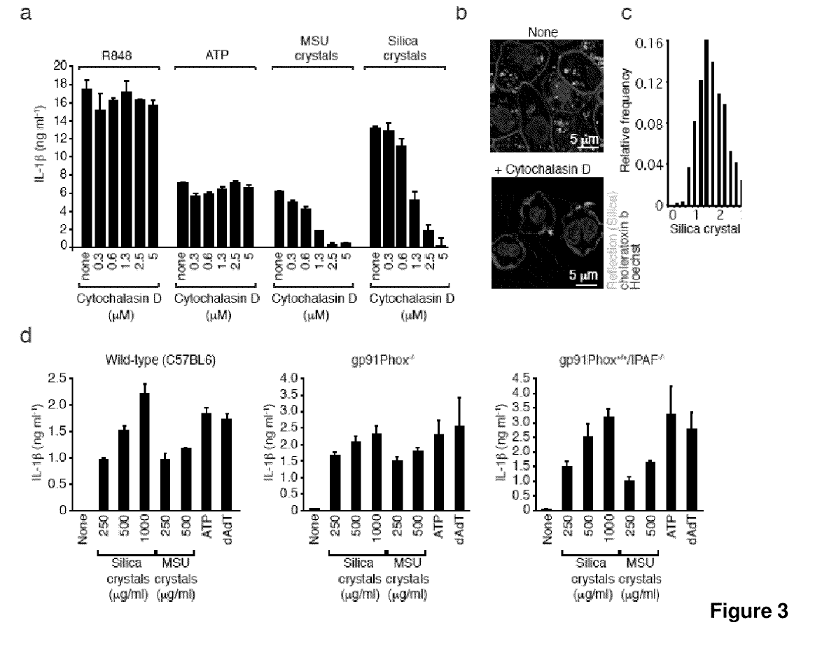

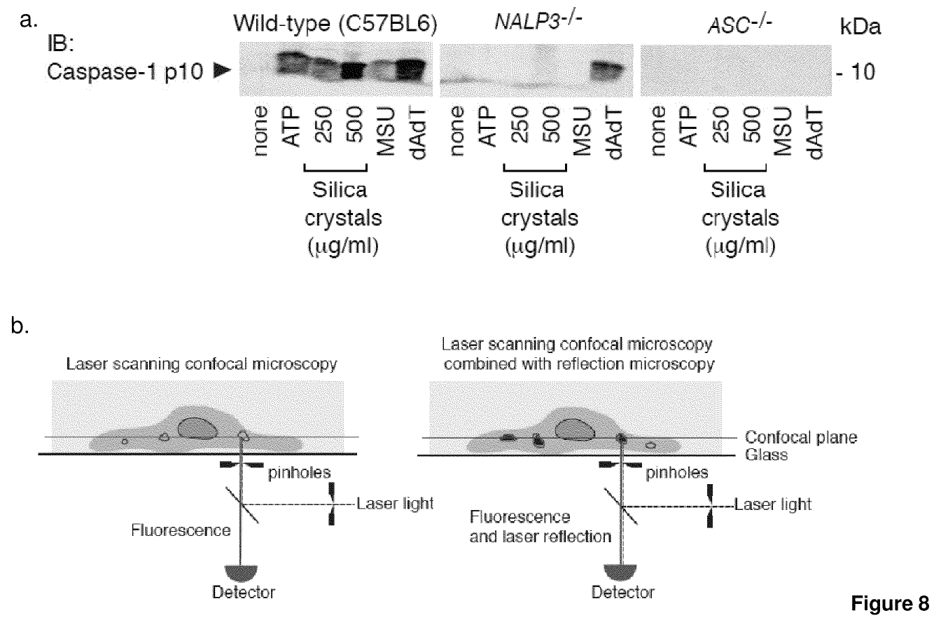

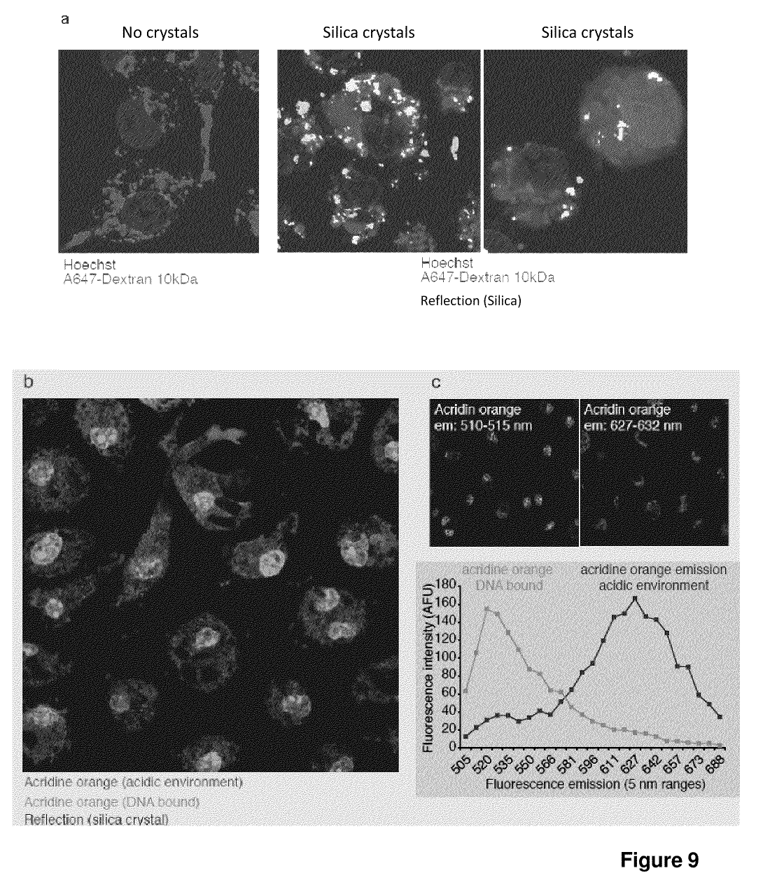

We next did imaging studies with macrophages undergoing phagocytosis of crystalline material to monitor the fate of the cargo engulfed. We used confocal reflection microscopy combined with fluorescence imaging to study the subcellular distribution of silica crystals over time (FIG. 8b). DQ ovalbumin is a useful tool for monitoring the endo-lysosomal compartment in living cells Q8 in real time. The fluorescence of the fluorophore BODIPY-FL (8-chloromethyl-4,4-difluoro-1,3,5,7-tetramethyl-4-bora-3a,4a-diaza-sindacene) on DQ ovalbumin is normally quenched unless the protein is proteolytically processed into peptides in endo-lysosomal compartments. In untreated cells, processed DQ ovalbumin was localized to small vesicular and tubular endosomes or lysosomes, as expected. In contrast, we detected large swollen lysosomes in most cells exposed to silica crystals. Many cells had a cytosolic pattern of fluorescently processed DQ ovalbumin, which indicated lysosomal rupture or leakage of lysosomal contents into the cytosol of crystal-treated cells (FIG. 4a). Fluorescent indicator dyes that stain acidic environments confirmed those findings (data not shown). In addition, ingested fluorescent dextran, which traffics into the lysosomal pathway, also stained swollen lysosomal compartments and showed cytosolic translocation in most cells (FIG. 9) .B6-MCLs were incubated with A647-Dextran for 30 min and then either left untreated or stimulated with silica crystals (250 μg/m1). Lysosomal swelling could be easily seen 90 min after incubation when cells were analyzed by confocal microscopy. Such swelling of lysosomes was not exclusive to silica crystal-treated cells, as MSU crystals elicited similar morphological changes and rupture of lysosomal compartments (data not shown).

We also tested if crystals could be detected in cells outside phagosomal compartments by staining the plasma membrane and phagosomal membranes of fixed and permeabilized cells with fluorescent cholera toxin B-subunit. Most crystals were located in phagosomes that had distinct membrane staining; however, some crystals were in regions were not associated with membranes and thus were translocated to the cytosol (FIG. 4b). We obtained similar data when we examined MSU crystals (data not shown). To quantify the degree of phagosomal rupture after crystal treatment, we used acridine orange, a dye used to measure lysosomal integrity. Acridine orange fluoresces green in its monomeric state and binds to nuclear and cytosolic DNA and RNA. Because of its cationic nature, acridine orange becomes highly concentrated in acidic compartments, resulting in the formation of dimers and the appearance of red fluorescence (FIG. 9). The amount of red fluorescence of acridine orange in lysosomes directly correlates with the amount of acidic lysosomes in cells. We took advantage of this property and monitored the lysosomal content of macrophages before and after treatment with silica crystals. Increasing amounts of silica crystals resulted in loss of lysosomes, as indicated by less red fluorescence of acridine orange (FIG. 4c, top). Furthermore, we found lysosomal rupture due to silica phagocytosis to a similar extent in cells deficient Q9 in NALP3 , which indicated that lysosomal damage was independent of NALP3 (FIG. 4c, bottom). Collectively, these data show that phagocytosis of crystalline material leads to active swelling of phagosomes, followed by phagosomal destabilization and rupture, thus resulting in the release of phagosomal contents, which then gain access to the cytosolic compartment.

Lysosomes contain a plethora of proteolytic enzymes, many of which are activated by acidification of lysosomal pathways. To assess the function of lysosomal acidification in silica-mediated NALP3 activation, we used bafilomycin A to block the vacuolar H+ ATPase system, which is required for the acidification of lysosomal compartments. As expected, bafilomycin A blocked the formation of acidic lysosomes in macrophages, as assessed with the ‘lysomotropic’ pH-activated dye LysoSensor Green, which fluoresces only after accumulation in acidic environments (FIG. 4d). In addition, bafilomycin A suppressed the rapid, dose-dependent appearance of fluorescent DQ ovalbumin (FIG. 4e), which indicated lower lysosomal proteolytic function. Notably, bafilomyin A completely blocked silicamediated IL-1β release but had no effect on ATP (FIG. 4f), which suggested a pivotal function for lysosomes in for crystal-mediated NALP3 activation.

### Example 6

### Crystal-induced Lysosomal Destabilization Triggers Inflammasome Activation

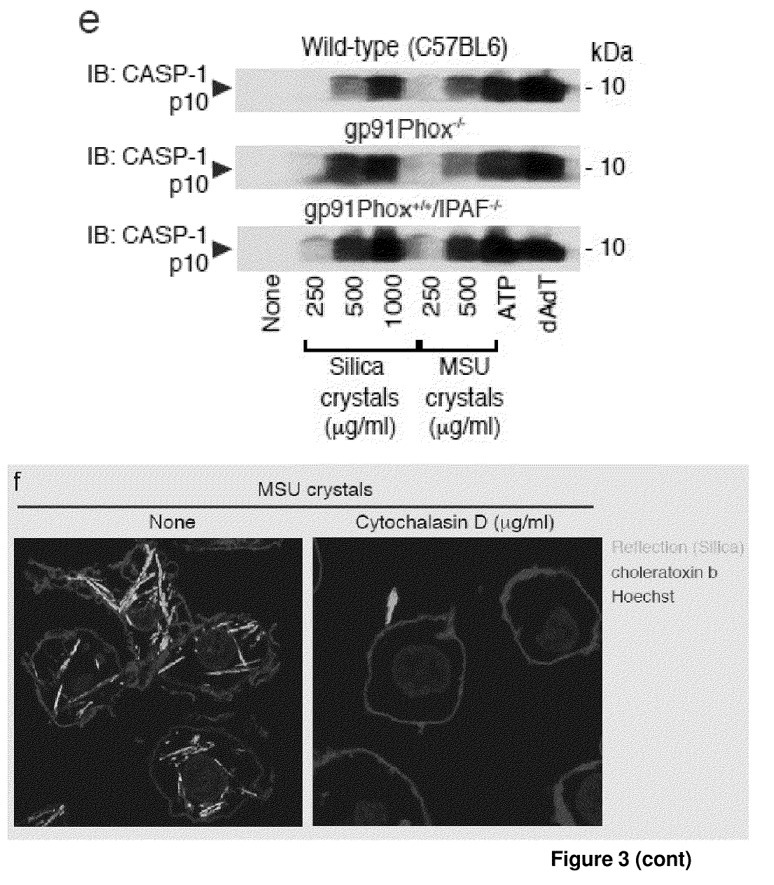

Because one important function of lysosomal acidification is the pH-dependent activation of proenzymes to further degrade lysosomal contents, we hypothesized that activation of lysosomal proenzymes could be a critical event in silica-mediated inflammasome activation. In particular, the cathepsin family of proenzymes was a likely candidate. To address their function in silica-mediated inflammasome activation, we tested several cathepsin inhibitors for their ability to affect silica-mediated IL-1β release. Among the inhibitors tested, CA-074-Me, a cathepsin B-specific inhibitor¹⁶, led to much less caspase-1 activation after silica crystal treatment (FIG. 5a). We also detected mature cathepsin B in supernatants of crystal-stimulated cells independently of NALP3 (FIG. 5b). To assess whether release of cathepsin B from lysosomes was temporally associated with caspase-1 Q11 activation, we incubated mouse macrophages with silica crystals and added fluorescent peptides that indicate cathepsin B activity by an increase in red fluorescence and caspase-1 activity by green fluorescence. When we incubated resting cells with those two reporter peptides, we found red fluorescence in lysosomal compartments, indicative of cathepsin B activity, whereas resting cells did not acquire green fluorescence because they lacked caspase-1 activity (FIG. 5c, left). Cells treated with silica crystals acquired either red fluorescence because of lysosomal containment of the cathepsin B-indicator peptide or green cytoplasmic fluorescence indicative of caspase-1 activity (FIG. 5c, right). Activated cells obtained from caspase-1-deficient macrophages did not stain with the caspase-1-indicator Q12 fluorescent peptide, which demonstrated the specificity of the reagent (data not shown). This notable difference between staining of either lysosomal cathepsin B or activated caspase-1 suggested that cells that lose lysosomal cathepsin B after lysosomal rupture (loss of red lysosomal fluorescence) rapidly activate caspase-1. These results collectively suggest that the NALP3 inflammasome senses lysosomal contents released into the cytosol of phagocytic macrophages after crystal-induced lysosomal damage.

### Example 7

### Alum Triggers NALP3 Activation Through Lysosomal Destabilization

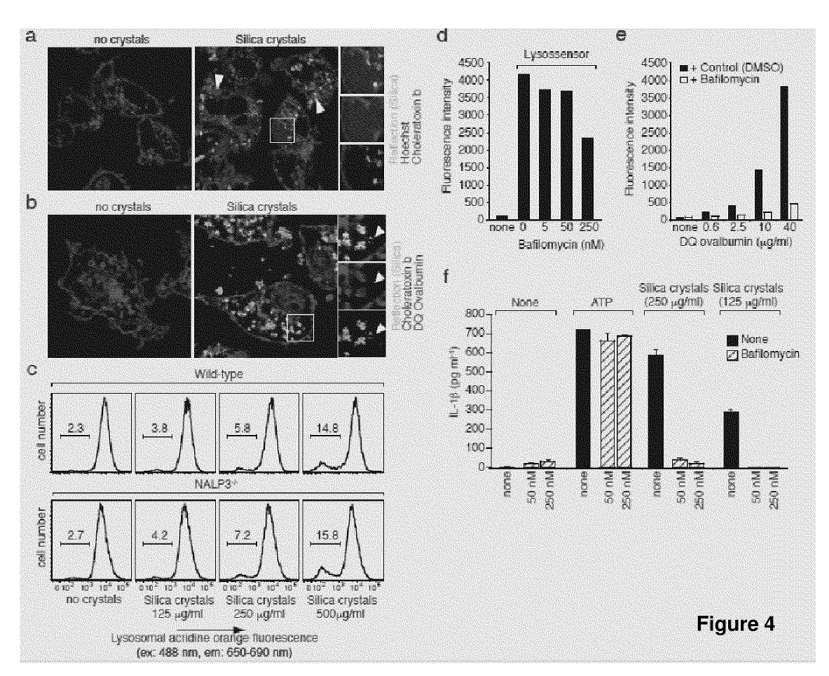

Aluminum salts (alum) are the most commonly used vaccine adjuvants, and all alum preparations contain crystals. Aluminum hydroxide has been reported to induce the cleavage of IL-1β and IL-18 in a caspase-1-dependent way¹⁷. To determine whether alum induces inflammation by a mechanism similar to that of silica crystals, we did experiments with a mixture of aluminum hydroxide and magnesium hydroxide (Imject alum). Alum induced IL-1β maturation and release by human PBMCs (FIG. 6a) in a caspase-1-dependent way (data not shown). In mouse macrophages, the alum-induced release of IL-1β was dependent on NALP3 and ASC (FIG. 6b), which indicated that alum triggers inflammation through activation of the NALP3 inflammasome. Additionally, the influx of neutrophils into the peritoneum after intraperitoneal administration of alum was dependent on IL-1 activity (FIG. 6c). Of note, alum added to cells induced considerable morphological changes and led to lysosomal rupture, as shown by the cytosolic translocation of DQ ovalbumin (FIG. 6d) and the lysosomal staining pattern of acridine orange (FIG. 6e). As noted with silica crystals, cells incubated with alum stained positively with a cathepsin B indicator or the caspase-1 indicator (FIG. 6f), which demonstrated close correlation of lysosomal loss and caspase-1 activation. In agreement with that idea, alum-induced release of IL-1β was partially dependent on lysosomal acidification and cathepsin B activity (FIG. 6g). Uricase added to crystal- or ATP-stimulated cells inhibited the MSU crystal-elicited release of IL-1β in a dose-dependent way but did not change the response to alum (FIG. 6h).

### Example 8

### NALP3 Activation by Crystal-independent Lysosomal Damage

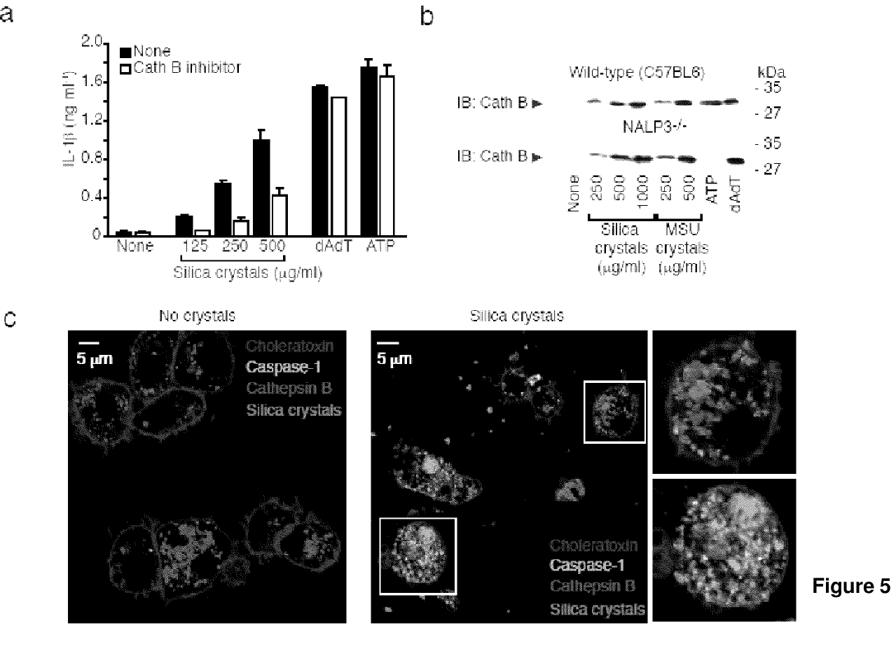

To determine if lysosomal rupture alone was sufficient to activate the inflammasome, we turned to a model with which we could assess the function of lysosomal rupture without the requirement of the addition of known inflammasome activators. We loaded mouse macrophages with a hypertonic solution and then placed them in hypotonic media, which resulted in the rupture of endo-lysosomal compartments. This strategy successfully translocated endo-lysosomal fluorescent dextran into the cytosol, as shown by diffuse cytosolic staining of cells after the treatment (FIG. 7a). After this crystal-independent disruption of lysosomes, there was caspase-1 cleavage (FIG. 7b, left) that was partially inhibited by a cathepsin B inhibitor. Notably, this crystal-independent lysosomal rupture was completely dependent on NALP3 (FIG. 7b, right). These results collectively indicate that rupture of lysosomal compartments and leakage of lysosomal contents into the cytosol even in the absence of crystal material was sufficient to trigger activation of the NALP3 inflammasome in a partially cathepsin Q13 B-dependent way. Additionally, we used THP-1 human monocytes differentiated in the presence of phorbol 12-myristate 13-acetate (PMA) in this lysosomal rupture assay; this represents a model system that does not rely on microbial stimuli for pro-IL-1β priming.

Similar to the findings with mouse macrophages, THP-1 cells were also activated to release cleaved IL-1β after lysosomal rupture in a partially cathepsin B-dependent way. THP-1 cells were incubated in the presence of fluorescent dextran (red) for 30 min and were left untreated or were subsequently treated using hypertonic and hypotonic solutions to induce lysosomal rupture, producing similar results to those described above. Also, THP-1 cells were incubated with CA-074-Me or left untreated and lysosomal rupturing was subsequently induced. Cath B inhibitor clearly reduced IL-1β release. In addition, untreated cells were either stimulated with ATP or left untreated. 4 h after stimulation, supernatants were assessed for IL-1β by ELISA and Western blot. ATP stimulated cells showed increased IL-1β release.

To confirm those findings, we used an alternative method to disrupt lysosomes with the ‘lysomotropic’ reagent Leu-Leu-OMe (L-leucyl-L-leucine methyl ester), which is known to induce lysosomal damage^{18,19.} Leu-Leu-OMe is a ‘functionalized’ dipeptide that is converted into a membrane-lysing compound by the lysosomal enzyme dipeptidyl peptidase I. We incubated mouse macrophages with Leu-Leu-OMe at the reported effective doses and noted considerable swelling and rupture of lysosomes in most cells, as indicated by lysosomal morphology, loss of red fluorescence of acridine orange and diffuse cytoplasmic staining by fluorescent dextran (FIG. 7c) Inhibition of lysosomal acidification by bafilomycin resulted in much less altered lysosomal morphology and rupture (data not shown). We next assessed whether Leu-Leu-OMe activated IL-1β maturation. We detected large quantities of IL-1β in cellular supernatants in response to Leu-Leu- OMe, similar to the concentrations elicited by known inflammasome activators (FIG. 7d). To test whether the Leu-Leu-OMe-induced lysosomal damage induced NALP3 activation, we analyzed macrophages from mice deficient in NALP3 and ASC. As noted with silica crystals, cells deficient in NALP3 and ASC failed to respond to Leu- Leu-OMe-induced lysosomal damage and released only small quantities of IL-1β (FIG. 7e, left). Finally, inhibition of lysosomal pH by bafilomycin or inhibition of cathepsin B also inhibited the release of IL-1b in response to Leu-Leu-OMe (FIG. 7e, right).

### REFERENCES FOR EXAMPLES 1-8

1. Mossman, B. T. & Churg, A. Mechanisms in the pathogenesis of asbestosis and silicosis. American journal of respiratory and critical care medicine 157, 1666-1680 (1998).

2. Huaux, F. New developments in the understanding of immunology in silicosis. Current opinion in allergy and clinical immunology 7, 168-173 (2007).

3. Martinon, F., Petrilli, V., Mayor, A., Tardivel, A. & Tschopp, J. Gout-associated uric acid crystals activate the NALP3 inflammasome. Nature 440, 237-241 (2006).

4. Petrilli, V., Dostert, C., Muruve, D. A. & Tschopp, J. The inflammasome: a danger sensing complex triggering innate immunity. Current opinion in immunology 19, 615-622 (2007).

5. Agostini, L., Martinon, F., Burns, K., McDermott, M. F., Hawkins, P. N. & Tschopp, J. NALP3 forms an IL-1β eta-processing inflammasome with increased activity in Muckle-Wells autoinflammatory disorder. Immunity 20, 319-325 (2004).

6. Dinarello, C. A. Interleukin-1 beta, interleukin-18, and the interleukin-1 beta converting enzyme. Annals of the New York Academy of Sciences 856, 1-11 (1998).

7. Muruve, D. A., Petrilli, V., Zaiss, A. K., White, L. R., Clark, S. A., Ross, P. J., Parks, R. J. & Tschopp, J. The inflammasome recognizes cytosolic microbial and host DNA and triggers an innate immune response. Nature 452, 103-107 (2008).

8. Ozinsky, A., Underhill, D. M., Fontenot, J. D., Hajjar, A. M., Smith, K. D., Wilson, C. B., Schroeder, L. & Aderem, A. The repertoire for pattern recognition of pathogens by the innate immune system is defined by cooperation between toll-like receptors. Proceedings of the National Academy of Sciences of the United States of America 97, 13766-13771 (2000).

9. Brown, G. D., Taylor, P. R., Reid, D. M., Willment, J. A., Williams, D. L., Martinez-Pomares, L., Wong, S. Y. & Gordon, S. Dectin-1 is a major beta-glucan receptor on macrophages. The Journal of experimental medicine 196, 407-412 (2002).

10. Mariathasan, S., Weiss, D. S., Newton, K., McBride, J., O'Rourke, K., Roose-Girma, M., Lee, W. P., Weinrauch, Y., Monack, D. M. & Dixit, V. M. Cryopyrin activates the inflammasome in response to toxins and ATP. Nature 440, 228-232 (2006).

11. Mariathasan, S., Newton, K., Monack, D. M., Vucic, D., French, D. M., Lee, W. P., Roose-Girma, M., Erickson, S. & Dixit, V. M. Differential activation of the inflammasome by caspase-1 adaptors ASC and Ipaf. Nature 430, 213-218 (2004).

12. Kool, M., Soullie, T., van Nimwegen, M., Willart, M. A., Muskens, F., Jung, S., Hoogsteden, H. C., Hammad, H. & Lambrecht, B. N. Alum adjuvant boosts adaptive immunity by inducing uric acid and activating inflammatory dendritic cells. The Journal of experimental medicine 205, 869-882 (2008).

13. Kanneganti, T. D., Ozoren, N., Body-Malapel, M., Amer, A., Park, J. H., Franchi, L., Whitfield, J., Barchet, W., Colonna, M., Vandenabeele, P., Bertin, J., Coyle, A., Grant, E. P., Akira, S. & Nunez, G. Bacterial RNA and small antiviral compounds activate caspase-1 through cryopyrin/NALP3. Nature 440, 233-236 (2006).

14. Fubini, B. & Hubbard, A. Reactive oxygen species (ROS) and reactive nitrogen species (RNS) generation by silica in inflammation and fibrosis. Free radical biology & medicine 34, 1507-1516 (2003).

15. Pollock, J. D., Williams, D. A., Gifford, M. A., Li, L. L., Du, X., Fisherman, J., Orkin, S. H., Doerschuk, C. M. & Dinauer, M. C. Mouse model of X-linked chronic granulomatous disease, an inherited defect in phagocyte superoxide production. Nature genetics 9, 202-209 (1995).

16. Buttle, D. J., Murata, M., Knight, C. G. & Barrett, A. J. CA074 methyl ester: a proinhibitor for intracellular cathepsin B. Archives of biochemistry and biophysics 299, 377-380 (1992).

17. Li, H., Nookala, S. & Re, F. Aluminum hydroxide adjuvants activate caspase-1 and induce IL-1β eta and IL-18 release. J Immunol 178, 5271-5276 (2007).

18. Thiele, D. L. & Lipsky, P. E. Regulation of cellular function by products of lysosomal enzyme activity: elimination of human natural killer cells by a dipeptide methyl ester generated from L-leucine methyl ester by monocytes or polymorphonuclear leukocytes. Proceedings of the National Academy of Sciences of the United States of America 82, 2468-2472 (1985).

19. Thiele, D. L. & Lipsky, P. E. Mechanism of L-leucyl-L-leucine methyl ester-mediated killing of cytotoxic lymphocytes: dependence on a lysosomal thiol protease, dipeptidyl peptidase I, that is enriched in these cells. Proceedings of the National Academy of Sciences of the United States of America 87, 83-87 (1990).

20. Kanneganti, T. D., Lamkanfi, M., Kim, Y. G., Chen, G., Park, J. H., Franchi, L., Vandenabeele, P. & Nunez, G. Pannexin-1-mediated recognition of bacterial molecules activates the cryopyrin inflammasome independent of Toll-like receptor signaling. Immunity 26, 433-443 (2007).

21. Marina-Garcia, N., Franchi, L., Kim, Y. G., Miller, D., McDonald, C., Boons, G. J. & Nunez, G. Pannexin-1-Mediated Intracellular Delivery of Muramyl Dipeptide Induces Caspase-1 Activation via Cryopyrin/NLRP3 Independently of Nod2. J Immunol 180, 4050-4057 (2008).

22. Takeda, K., Kaisho, T. & Akira, S. Toll-like receptors. Annual review of immunology 21, 335-376 (2003).

23. Petrilli, V., Papin, S., Dostert, C., Mayor, A., Martinon, F. & Tschopp, J. Activation of the NALP3 inflammasome is triggered by low intracellular potassium concentration. Cell death and differentiation 14, 1583-1589 (2007).

24. Franchi, L., Kanneganti, T. D., Dubyak, G. R. & Nunez, G. Differential requirement of P2X7 receptor and intracellular K+ for caspase-1 activation induced by intracellular and extracellular bacteria. The Journal of biological chemistry 282, 18810-18818 (2007).

25. Cain, K., Bratton, S. B. & Cohen, G. M. The Apaf-1 apoptosome: a large caspase-activating complex. Biochimie 84, 203-214 (2002).

26. Karki, P., Seong, C., Kim, J. E., Hur, K., Shin, S. Y., Lee, J. S., Cho, B. & Park, I. S. Intracellular K(+) inhibits apoptosis by suppressing the Apaf-1 apoptosome formation and subsequent downstream pathways but not cytochrome c release. Cell death and differentiation 14, 2068-2075 (2007).

27. Hentze, H., Lin, X. Y., Choi, M. S. & Porter, A. G. Critical role for cathepsin B in mediating caspase-l-dependent interleukin-18 maturation and caspase-l-independent necrosis triggered by the microbial toxin nigericin. Cell death and differentiation 10, 956-968 (2003).

28. Pelegrin, P. & Surprenant, A. Pannexin-1 mediates large pore formation and interleukin-lbeta release by the ATP-gated P2X7 receptor. The EMBO journal 25, 5071-5082 (2006).

29. Pelegrin, P. & Surprenant, A. Pannexin-1 couples to maitotoxin- and nigericin-induced interleukin-lbeta release through a dye uptake-independent pathway. The Journal of biological chemistry 282, 2386-2394 (2007).

30. Fujisawa, A., Kambe, N., Saito, M., Nishikomori, R., Tanizaki, H., Kanazawa, N., Adachi, S., Heike, T., Sagara, J., Suda, T., Nakahata, T. & Miyachi, Y. Disease-associated mutations in CIASI induce cathepsin B-dependent rapid cell death of human THP-1 monocytic cells. Blood 109, 2903-2911 (2007).

31. Willingham, S. B., Bergstralh, D. T., O'Connor, W., Morrison, A. C., Taxman, D. J., Duncan, J. A., Barnoy, S., Venkatesan, M. M., Flavell, R. A., Deshmukh, M., Hoffman, H. M. & Ting, J. P. Microbial pathogen-induced necrotic cell death mediated by the inflammasome components CIASI/cryopyrin/NLRP3 and ASC. Cell host & microbe 2, 147-159 (2007).

32. Asagiri, M., Hirai, T., Kunigami, T., Kamano, S., Gober, H. J., Okamoto, K., Nishikawa, K., Latz, E., Golenbock, D. T., Aoki, K., Ohya, K., Imai, Y., Morishita, Y., Miyazono, K., Kato, S., Saftig, P. & Takayanagi, H. Cathepsin K-dependent toll-like receptor 9 signaling revealed in experimental arthritis. Science (New York, N.Y 319, 624-627 (2008).

33. Latz, E., Schoenemeyer, A., Visintin, A., Fitzgerald, K. A., Monks, B. G., Knetter, C. F., Lien, E., Nilsen, N. J., Espevik, T. & Golenbock, D. T. TLR9 signals after translocating from the ER to CpG DNA in the lysosome. Nature immunology 5, 190-198 (2004).

34. Latz, E., Verma, A., Visintin, A., Gong, M., Sirois, C. M., Klein, D. C., Monks, B. G., McKnight, C. J., Lamphier, M. S., Duprex, W. P., Espevik, T. & Golenbock, D. T. Ligand-induced conformational changes allosterically activate Toll-like receptor 9. Nature immunology 8, 772-779 (2007).

35. Dostert, C., Petrilli, V., Van Bruggen, R., Steele, C., Mossman, B. T. & Tschopp, J. Innate Immune Activation Through NALP3 Inflammasome Sensing of Asbestos and Silica. Science (New York, N.Y (2008).

36. Franchi, L., Amer, A., Body-Malapel, M., Kanneganti, T. D., Ozoren, N., Jagirdar, R., Inohara, N., Vandenabeele, P., Bertin, J., Coyle, A., Grant, E. P. & Nunez, G. Cytosolic flagellin requires Ipaf for activation of caspase-1 and interleukin lbeta in salmonella-infected macrophages. Nature immunology 7, 576-582 (2006).

37. Schiltz, C., Liote, F., Prudhommeaux, F., Meunier, A., Champy, R., Callebert, J. &

Bardin, T. Monosodium urate monohydrate crystal-induced inflammation in vivo: quantitative histomorphometric analysis of cellular events. Arthritis and rheumatism 46, 1643-1650 (2002).

38. Severa, M., Coccia, E. M. & Fitzgerald, K. A. Toll-like receptor-dependent and -independent viperin gene expression and counter-regulation by PRDI-binding factor-1/BLIMPI. The Journal of biological chemistry 281, 26188-26195 (2006).

39. Roberson, S. M. & Walker, W S Immortalization of cloned mouse splenic macrophages with a retrovirus containing the v-raf/mil and v-myc oncogenes. Cellular immunology 116, 341-351 (1988).

The following Examples 9 to 14 illustrate the ability of aggregated β-amyloid peptide to stimulate the inflammasome and the release of IL-1β.

Materials and Methods for Example 9 to 14:

Mice:

NALP3^{-/-28}, ASC^{-/-28} and IPAF^{-/-}mice³⁸ were from Millennium Pharmaceuticals (Cambridge, Mass.). MyD88^{-/-}and Caspase-1^{-/-39} mice were kindly provided by Shizuo Akira (University of Osaka, Japan) and Amy Hise (Case Western Reserve University, Cleveland), respectively. Cathepsin B^{-/-} mice were described previously⁴⁰. C57BL/6 mice and IL-1R^{-/-} were from Jackson Laboratories (Bar Harbor, Me.). All mouse strains were housed and bred under pathogen-free conditions. All experiments were performed in accordance with the guidelines set forth by the University of Massachusetts Medical School Department of Animal Medicine and the Institutional Animal Care and Use Committee.

Reagents:

ATP, cytochalasin D, LPS, pepstatin A, poly-L-lysine, bovine serum albumin, poly(deoxyadenylic-thymidylic) acid sodium salt (dAdT), saponin and zymosan were purchased from Sigma-Aldrich (St. Louis, MO). CA-074-Me and Z-FF-FMK were from Calbiochem (Gibbstown, N.J.). Goat anti-mouse Cathepsin B antibody was from R&D Systems (Minneapolis, Minn.), mouse anti mammalian TUJ-1 antibody from Covance (Berkeley, Calif.) and rat anti mouse CD11b and rat anti mouse F4/80 antibodies from Serotec (Raleigh, N.C.). Biotin-labeled rat anti mouse CD 107A antibody (LAMP-1) was from eBioscience (San Diego, Calif.). Polyclonal rabbit anti-glial fibrillary acidic protein (GFAP) was from DAKO (Carpinteria, Calif.). A647-conjugated goat anti rabbit, A568-conjugated streptavidin, A488-conjugated goat anti mouse, A568-conjugated donkey anti goat, A647-conjugated and A488-conjugated goat anti rat antibodies, A647-conjugated choleratoxin B, LysoTracker green, red and Hoechst 33258 were obtained from Invitrogen (Carlsbad, Calif.). Recombinant mouse IFNγ was from BD Pharmingen (San Jose, Calif.). Paraformaldehyde was from Electron Microscopy Sciences (Hatfield, Pa.). Caspase-1 inhibitor z-YVAD-fmk was purchased from BioVision Research Products (Mountain View, Calif.).

β-Amyloid:

Aβ 1-42 peptide, HiLyte 488-conjugated Aβ 1-42, and revAβ (control non-fibrillary peptide with identical primary structure in reverse order), were from Anaspec (San Jose, Calif.) or from American Peptide Company (Sunnyvale, Calif.) and prepared as described⁴¹. Aβ preparations were tested for endotoxin using a Limulus Amebocyte Lysate (LAL) Assay (Associates of Cape Cod Incorporation, E. Falmouth, Mass.) and were found to be below detection limit A3-FITC was prepared according to the manufacturer's instructions (Invitrogen). Unbound FITC was removed by dialysis overnight in cold PBS.

Cell culture:

For primary cultures, bone marrow-derived macrophages and microglia were isolated as described^{42,43} and cultured in DMEM supplemented with L-glutamine, ciprofloxacin (Cellgro, Manassas, Va.) and 10% fetal calf serum (Hyclone, Logan, Utah) Immortalized macrophage and microglial cell lines were generated using J2 recombinant retrovirus (carrying v-myc and v-raf oncogenes)^{44,45}. Briefly, primary bone marrow cells were incubated in L929 conditioned medium for 3-4 days to induce macrophage differentiation. Cells were then infected with J2 recombinant retrovirus and maintained in culture until cells were growing in the absence of conditioned medium. Similarly, primary mixed glial cultures were cultured until fully confluent. After two consecutive infections with J2 retrovirus, cells were maintained in normal medium until growth of microglial cells in colonies was observed. The semi-adherent colonies of microglial cells were washed off and cultured in new flasks. Microglial cells lines and macrophage cell lines were extensively tested Immortalized microglial cells showed 100% purity and revealed high similarity in morphology and surface marker expression compared to primary microglia (Supplementary FIG. 1a-b). Microglial cell lines form wild type (C57/BL6), Caspase-1^{-/-} and IL-IR^{-/-} mice as well as macrophage cell lines from wild type (C57BL6), NALP3 -, IPAF- and ASC-deficient mice were generated and used for experiments as indicated.

Stimulations were carried out in serum free DMEM medium. Microglial cells and macrophages were primed with 100 U/ml IFNγ (Griess assay, TNF ELISA, real-time quantitative PCR) or 100 ng/ml ultrapure LPS (IL-1β ELISA, FLICA, ASC-CFP microglia cell line assay, Western blot) 1-3 h prior to stimulation with Aβ, revAβ, zymosan, ATP or transfection with dAdT.

ASC-CFP microglia cell line:

Human ASC was fused to CFP using BgIII/BamHI in pEF-BOS-CFP. Subsequently, ASC-CFP was cloned using XhoI and NotI into a modified form of FUGW (FUGW-XN). FUGW-XN was modified by replacing GFP with a novel cut site that created XhoI and NotI compatible ends upon Esp3I digestion.

Lentivirus was produced in 293T cells transfected with the FUGW based expression vector coding for ASC-CFP, lentiviral packaging plasmid containing gag, pol and rev genes (pCMV-dR8.91) and envelope plasmid (VSV-G) using TransIT-LT1 transfection reagent (Minis Bio, MIR 2300/5/6) as previously described⁴⁶. One day after transfection, medium was replaced and after one additional day of culture supernatants containing the lentivirus was removed, filtered and used to infect immortalized microglia cells.

For experiments, ASC-CFP microglial cells were seeded in duplicate into confocal dishes, stimulated as indicated. A647-labeled cholera toxin subunit B (1:1000) was added to stain cellular membranes. Cells were subsequently imaged using confocal microscopy (Leica SP2 AOBS). Cells were fixed and 5 random fields were imaged in duplicate for quantification.

Neurotoxicity and caspase I assay:

For assessment of microglia-induced neurotoxicity, CAD mouse neuronal cells⁴⁷ were seeded on poly-L-lysine coated glass cover slips and grown in F12/DMEM supplemented with ciprofloxacin (Cellgro, Manassas, Va.) and 10% fetal calf serum (Hyclone, Logan, Utah) for 24 h. Differentiation was induced by withdrawal of serum for 48 h Immortalized microglia from wildtype or caspase-1^{-/-} mice were added in serum free medium to obtain mixed neuronal/microglial cultures and immediately stimulated. 72 h after stimulation with Aβ or media, cells were fixed, stained for the neuron-specific marker TuJ-1 and microglial marker CD11b, and imaged by confocal microscopy (Leica SP2 AOBS). Caspase-1 activity in microglial cells was assessed using the caspase-1 FLICA™ kit (Immunochemistry Technologies, Bloomington, Minn.) according to the manufacturer's instructions.

ELISA and Nitric Oxide Measurement:

Cell culture supernatants were assayed for IL-1β and TNFα using ELISA kits from R&D Systems (Minneapolis, Minn.) according to the manufacturer's instructions. Nitric oxide was measured by Griess reaction of nitrites accumulated in the supernatants using chemicals from Sigma.

Immunocytochemistry:

Neuronal, microglial, mixed neuronal/microglial and primary mixed glial cultures were fixed with 4% paraformaldehyde for 30 min and incubations with respective primary and secondary antibodies were performed for 2 h at room temperature in PBS containing 1% bovine serum albumin, 5% fetal calf serum and 0.05% saponin Cell nuclei were stained with Hoechst 33258 (1:1000).

Stereotaxic brain microinjections:

Injections were performed as described²⁷. Briefly, six week old mice (n=4 in each group) were anesthetized with ketamine (100 mg/kg i.p.; Webster) and xylazine (10 mg/kg i.p.; Webster), placed on a homoeothermic heating blanket (Harvard Apparatus) and immobilized in a stereotaxic frame (Stoelting). A linear skin incision was made under sterile conditions, and a small hole was created in the skull using a dental drill 1 mm anterior and 2 mm lateral to bregma over both hemispheres. 1 μl Aβ in sterile pyrogen-free saline (1 mg/ml) was inoculated on the left side, and the same amount of revAβ was inoculated on the right side, using separate Hamilton syringes. Mice were allowed to fully recover on a heating blanket. After 48 hours, the mice were anesthetized with ketamine/xylazine and transcardially perfused with 4% paraformaldehyde in PBS. Brains were removed, placed in 4% PFA overnight at 4° C., and then transferred to PBS and stored at 4° C. until sectioning. Coronal 60 μm sections were taken using a vibratome (Leica). Antigen retrieval was performed by incubating slices in protease K (Invitrogen; 20 μg/ml in TRIS-EDTA buffer pH 8.0) for 6 min Slices were blocked in PBS containing 0.3% Triton-X100 (Fisher), 10% horse serum and 1% bovine serum albumin for 1 h, and subsequently incubated with primary anti-F4/80 antibody (rat; 1:100; MCA497R, Serotec) in 5% horse serum and 0.05% Triton-X100 in PBS for 24 h at room temperature. Staining was visualized by incubation with Alexa 488-conjugated goat anti-rat secondary antibody (1:500; Invitrogen) at room temperature for 3 h. Cell nuclei were stained with Hoechst 33258 (1:1000). Images were taken with a confocal microscope (Zeiss). To quantify the number of microglia recruited to sites of microinjection, serial sections from each site of injection were visualized by fluorescence microscopy (Nikon Eclipse E400) and digitally photographed. Microscope settings were the same for all experiments. Fluorescence intensity of microglia immunoreactive for F4/80 at each injection site was quantified using ImageJ (NIH).

Flow Cytometry Analysis:

For evaluation of lysosomal damage, cells were incubated with LysoTracker green (1:2000) for 30 min and stimulated as described. Lysosomal damage was defined as loss of LysoTracker fluorescence as assessed by flow cytometry. All flow cytometry experiments were performed on an LSRII cytometer (BD Biosciences), data were acquired by DIVA software (BD Biosciences) and analyzed with FlowJo software (Tree Star Inc., Ashland, Oreg.).

Quantitative Real-Time PCR (QRT-PCR):

Immortalized murine microglia were seeded in 6 well tissue culture plates, incubated in the absence or presence of 10 μM Aβ, washed with PBS and lysed in 1 mL TRIZOL reagent (Invitrogen). RNA was purified by organic extraction. 5 μg of RNA from each sample was reverse transcribed using multiscribe reverse transcriptase (Applied Biosystems). Oligonucleotide primers were designed using the Primer3 Input PCR primer design program (version 0.4.0.) and QRT-PCR experiments were performed using an iQ Real-Time PCR Detection System (Bio-Rad) and Optical System Software (Version 2.0, Bio-Rad). Relative quantification of mRNA expression was calculated by the comparative cycle method as described by the manufacturer.

Western Blot Analysis:

Cell culture supernatants were precipitated by adding an equal volume of methanol and 0.25 volumes of chloroform, vortexed and centrifuged at 20,000×g for 10 min. The upper phase was discarded and 500 μl of methanol was added to the interphase. This mixture was centrifuged at 20,000×g for 10 min and the protein pellet dried at 55° C., resuspended in Laemmli buffer and boiled at 99° C. for 5 min Samples were separated by SDS-PAGE (15%) and transferred onto nitrocellulose membranes. Blots were incubated with rabbit polyclonal antibody to anti murine caspase-1 p10 (sc-514, Santa Cruz Biotechnology, Santa Cruz, Calif.).

### Example 9

### Fibrillar β-Amyloid Induces Caspase-1-dependent Release of Interleukin-1β from Microglia

Activated microglia in senile plaques show increased expression of IL-1β⁸, and elevated levels of IL-1β are found in the cerebrospinal fluid of Alzheimer patients⁹. To investigate the mechanisms of Aβ-triggered IL-1β maturation and release in vitro, we incubated primary mouse microglial cells with fibrillar Aβ and the reverse, non-fibrillar Aβ (revAβ). Because pro-IL-1β is not constitutively expressed and requires transcriptional induction, cells were primed with LPS, similarly to previous studies¹¹⁻¹³, to ensure robust pro-IL1 induction and to mimic the chronic activation of microglia in the plaque-bearing brain²⁻⁴.

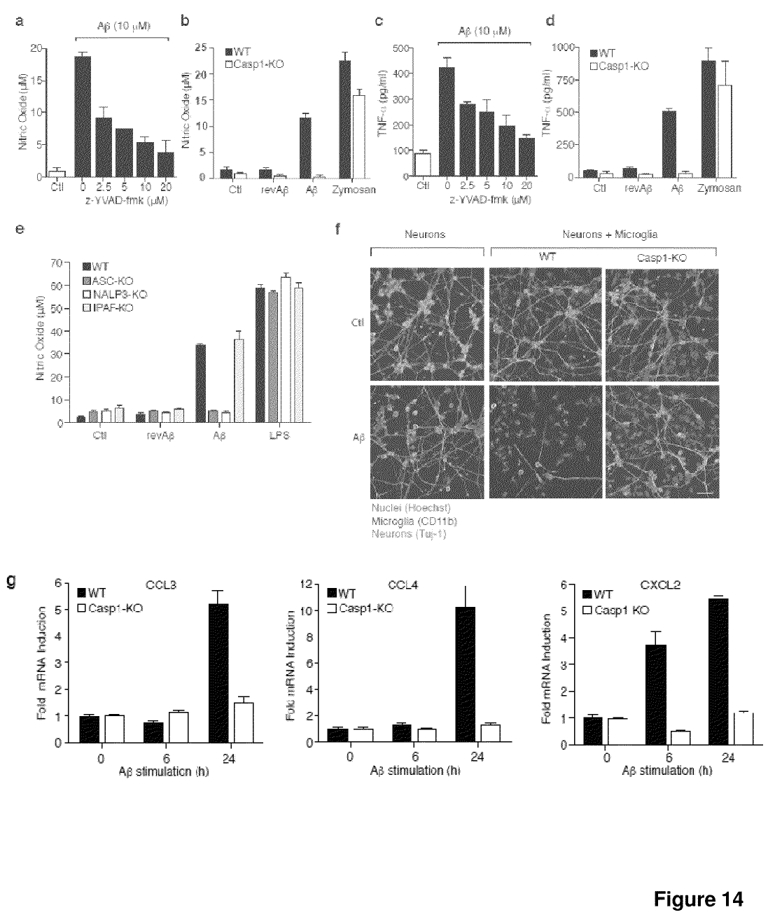

We found that fibrillar Aβ but not revAβ led to an early and pronounced IL-1β release into the supernatants (FIG. 10a). A similar rate of IL-1β release was found in a mouse microglial cell line that we established from wild-type C57/BL6 mice (FIG. 10b). These immortalized microglial cell lines are remarkably similar to primary mouse microglial preparations in morphology, expression of cell surface markers and function (FIG. 15a,b).

IL-1β maturation is controlled by caspase-1 after assembly of the inflammasome, a multi protein complex that activates pro-caspase-1. In order to test whether Aβ activates caspase-1, we measured levels of activated caspase-1 using a fluorescent cell permeable probe (FAM-YVAD-Fmk), which covalently binds only to activated caspase-1 (Fluorescent labeled Inhibitors of Caspases, FLICA)¹⁴. Confocal microscopy as well as flow cytometry analysis revealed a robust increase of caspase-1 positive microglial cells after stimulation with Aβ, as well as after stimulation with the NALP3 inflammasome activator ATP, whereas cells stimulated with revAβ showed no such response (FIG. 10c). Similar results were obtained in primary microglial cultures (FIG. 15c).

Furthermore, western blot analysis of caspase-1 revealed that Aβ induced caspase-1 cleavage (p10) at 10 μM, as did the positive controls ATP and transfected double stranded DNA (dAdT), whereas reverse Aβ at the same concentration initiated no specific caspase-1 cleavage (FIG. 10d). Finally, when wild type immortalized microglial cells were stimulated with Aβ in the presence of a caspase-1 specific inhibitor z-YVAD-fmk, we observed dose-dependent inhibition of IL-1β release (FIG. 10e). This effect was also observed in primary microglia (FIG. 15d).

Together, these data indicate that Aβ-induced release of IL-1β from microglia is mediated by activated caspase-1.

### Example 10

### Fibrillar (3-Amyloid Activates the NALP3 Inflammasome

Next, we sought to determine the pathway responsible for caspase-1 activation and subsequent IL-1β release. Specifically, we investigated whether the NALP3 inflammasome and apoptosis-associated speck-like (ASC) protein, an inflammasome adaptor protein involved in procaspase-1 autocatalysis¹⁵, are required.

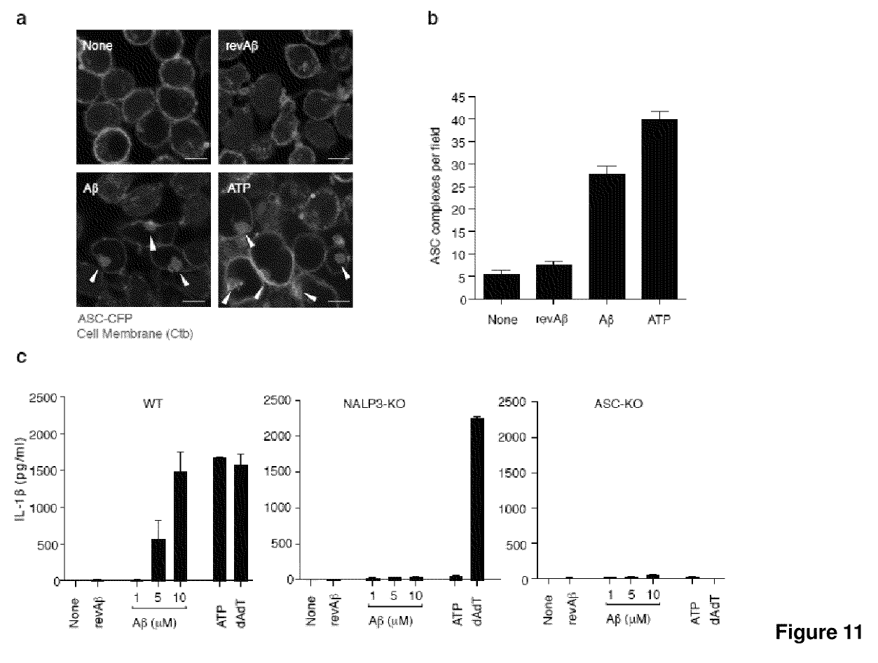

First, we stably transduced microglial cell cultures with a fusion protein of ASC and cyan fluorescent protein (CFP). ASC forms large oligomers upon its activation, leading to visible changes in the cytoplasmic fluorescence pattern of ASC-CFP. The clustering of ASC-CFP can be used as an optical reporter of ASC activation¹⁶. We found that under baseline conditions, ASC-CFP fluorescence was evenly distributed in the cytoplasm and nucleus (FIG. 11a). 3 h after stimulation with Aβ, brightly fluorescent clusters of ASC-CFP formed in the cytoplasm of many cells (FIG. 11a-b). These clusters were also observed after stimulation with ATP (FIG. 11a-b), but not with revAβ or LPS alone (FIG. 11a-b), indicating they represent cytoplasmic aggregates of activated ASC.

To investigate whether Aβ specifically activates the NALP3 inflammasome, we used bone marrow-derived macrophages from mice deficient in NALP3 or ASC. We observed strong and dose-dependent IL-1β release from macrophages of wild type mice (FIG. 11c), while macrophages from mice deficient in NALP3 or ASC failed to release IL-1β after stimulation with Aβ (FIG. 11c). These results show that Aβ activates the NALP3 inflammasome, resulting in caspase-1 activation and subsequent IL-1β maturation and release.

### Example 11

### Phagocytosis of (3-Amyloid is Important for Microglial IL-1β Production and Causes Lysosomal Damage in Microglia

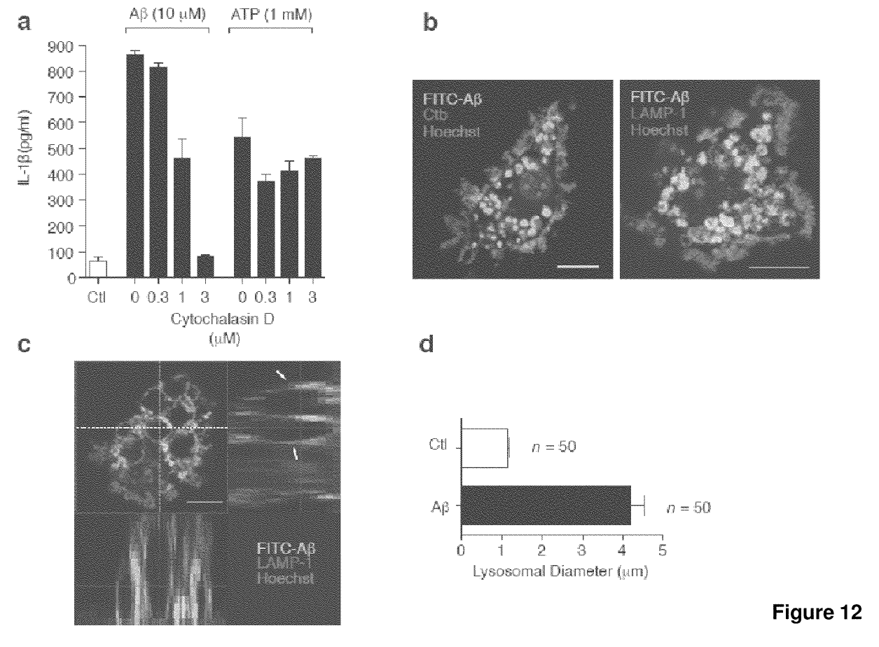

We were next interested in exploring the mechanism by which Aβ activates the NALP3 inflammasome. The NALP3 inflammasome is known to be activated by bacterial toxins, and several crystals and their phagocytosis have been shown to be important for NALP3 inflammasome activation¹³. Microglia are known to phagocytose Aβ in vitro and in vivo³. We therefore investigated whether phagocytosis was required for Aβ-induced IL-1β release. We incubated microglial cells with cytochalasin D, an inhibitor of Aβ-phagocytosis¹⁷, prior to and during stimulation with Aβ. We found that Aβ-induced IL-1β release from microglia was strongly attenuated by cytochalasin D (FIG. 12a), indicating that phagocytosis is required for IL-1β induction by Aβ. Cytochalasin D had no effect on IL-1β release after stimulation with ATP (FIG. 12a).

Next, we investigated phagocytosis of Aβ labeled with fluorescein isothyocyanate (FITC) Immunocytochemistry revealed that after 3 h, the majority of Aβ-FITC was internalized by microglia into discrete large intracellular compartments (FIG. 12b, left image). We identified these as lysosomes, as their membranes stained positive with the lysosomal marker protein LAMP-1 (FIG. 12b, right image). Confocal z-series analysis revealed that Aβ-containing lysosomes showed extensive enlargement and swelling in numerous cells (˜15% of all cells; FIG. 2c). We observed Aβ-containing lysosomes of up to 11 μm in diameter (mean, 4.1±0.4 μm), i.e. significantly larger than intact microglial lysosomes, which were 1.1±0.05 μm on average (FIG. 12d). These results show that Aβ is rapidly phagocytosed by microglia, and that lysosomal swelling, and potential dysfunction, occurs in the course of internalization.

To further explore whether lysosomal damage occurs during Aβ phagocytosis, we simultaneously monitored lysosomal integrity, and phagocytosis of fluorescently labeled Aβ using confocal microscopy. Intact lysosomes were identified by their accumulation of a lysomotropic dye (LysoTracker red), which is fluorescent at acidic pH levels. Live confocal microscopy showed that LysoTracker-positive lysosomes rapidly accumulated fibrillar Aβ (FIG. 12e). Shortly after incubation with Aβ, lysosomal size increased (FIG. 12d) as described above. Notable, these enlarged lysosomes, although readily identifiable by their accumulation of Aβ or by contrast interference imaging, did not enrich LysoTracker red (FIG. 12e), indicating that acidification and lysosomal function was compromised in enlarged Aβ-containing lysosomes. Consistent with this visual analysis, flow cytometric quantification of LysoTracker accumulation in microglial cultures revealed that with increasing amounts of Aβ, the percentage of cells that were negative for Lysotracker fluorescence increased dose-dependently (FIG. 12f). These data indicate that Aβ leads to lysosomal swelling, destabilization and dysfunction.

### Example 12

### Lysosomal Damage Triggers Cathepsin B Release, Which is Involved in Microglial Activation

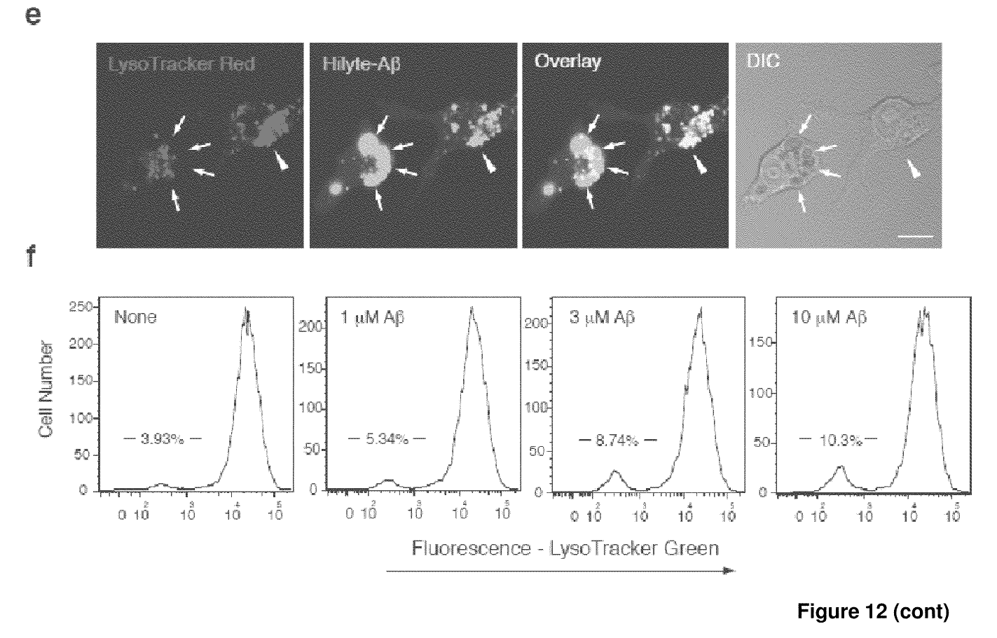

We next explored the possibility that release of lysosomal factors into the cytosolic compartment, rather than direct actions of Aβ, might be responsible for subsequent activation of the NALP3 inflammasome and caspase-1. Lysosomes contain many proteolytic enzymes, including the cathepsin family. Cathepsin B, one of its members, has previously been linked to the pathogenesis of Alzheimer's disease. Increased levels of cathepsin B in microglia surrounding senile plaques have been reported¹⁸, and cathepsin B inhibition proved therapeutically beneficial in a mouse model of Alzheimer's disease¹⁹. We therefore investigated whether cathepsin B is released following Aβ-induced lysosomal damage in microglia. Using an antibody against cathepsin B, we found that in non-stimulated cells, intracellular cathepsin B shows the expected punctuated staining pattern, consistent with its localization in lysosomes (FIG. 13a, left column). Early after phagocytosis of Aβ-FITC, cathepsin B co-localized with Aβ in small, i.e. structurally intact, lysosomes (FIG. 12h, middle column). However, 4 h after Aβ stimulation, lysosomes of 15-20% of cells were enlarged and did not stain for cathepsin B (FIG. 13a, right column). Notably, the overall cellular staining pattern of cathepsin B appeared diffuse and less punctuate (FIG. 13a, right column) and was outside of LAMP-1-positive enlarged lysosomes (FIG. 13a, inset), indicating release of cathepsin B from damaged lysosomes into the cytoplasm.

To investigate whether cathepsin B activity following stimulation with Aβ is functionally linked to microglial activation, we measured the effect of cathepsin B inhibition on microglial IL-1β release. We found that CA-074-Me, a specific inhibitor of cathepsin B²⁰ dose-dependently inhibited microglial IL-1β release (FIG. 13b), whereas pepstatin A and Z-FF-FMK, inhibitors of cathepsin D and cathepsin L, respectively, had no effect (FIG. 13b). Moreover, all three cathepsin inhibitors at their highest concentration did not inhibit IL-1β release when microglia were stimulated with ATP (FIG. 13b). Consistently, IL-1β release was strongly reduced in cathepsin B^{-/-} cells compared to wildtype cells after stimulation with Aβ at different concentrations, whereas IL-1β release was similarly strong after stimulation with ATP and dAdT in cathepsin B^{-/-} and wildtype cells (FIG. 13c). These data indicate that the effect of Aβ on IL-1β release were specifically linked to cathepsin B, and not related to effects on general cell physiology.

To investigate whether cathepsin B-induced release of IL-1β occurs via activation of caspase-1, and not by caspase-1-independent mechanisms, we directly quantified caspase-1 activation in microglia using FLICA assay. Flow cytometry revealed that caspase-1 activation after stimulation with Aβ was strongly and dose-dependently inhibited by the cathepsin B inhibitor Ca-074-Me (FIG. 13d). The induction of caspase-1 activation was not affected by cathepsin B inhibition when cells were stimulated with ATP (FIG. 13d), again underscoring the specific link between Aβ and cathepsin B.

Overall, these results indicate that microglial phagocytosis of Aβ induces lysosomal enlargement and loss of lysosomal integrity leading to release of lysosomal content into the cytoplasm. Furthermore, the release of specific proteases appears to be causally related to inflammasome activation.

### Example 13

### β-Amyloid-induced Expression of Pro-inflammatory and Chemotactic Factors Are Mediated by Caspase-1 Activation in Microglia

Microglia surrounding Aβ-containing senile plaques acquire an activated morphology and secrete chemotactic and pro-inflammatory molecules⁴, which contribute to the recruitment of microglia and may amplify the neurotoxic effects of Aβ. We asked whether activation of caspase-1 is involved in Aβ-induced secretion of these factors.

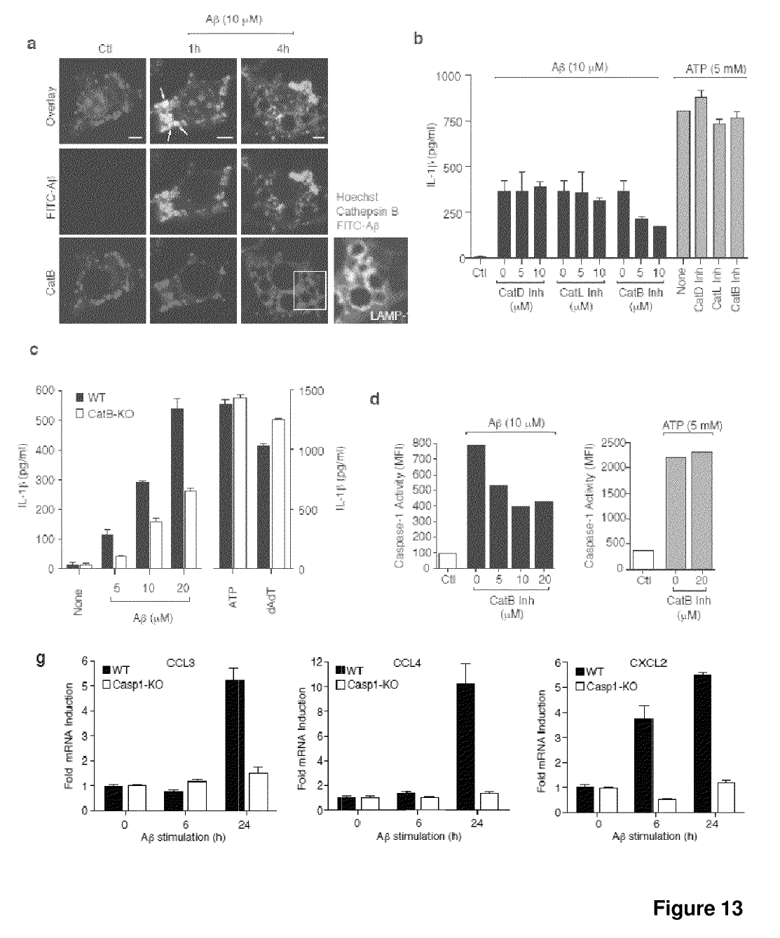

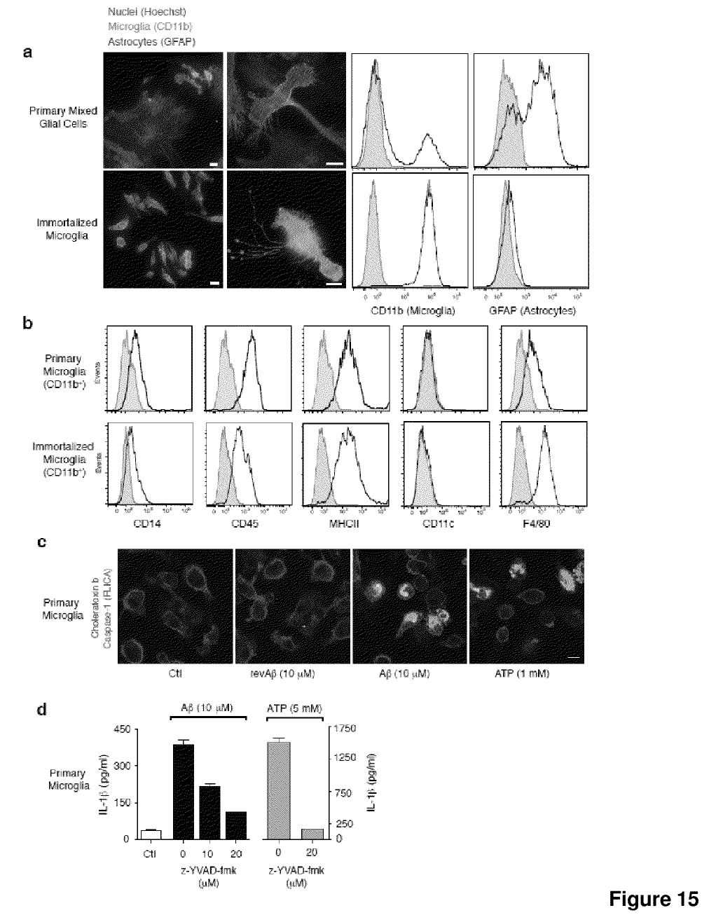

First, we studied the release of nitric oxide (NO) and TNF-α from microglia, two important mediators of Aβ-induced neurotoxicity and inflammation^{21,22}. NO synthesis in immortalized microglial cultures was strongly and dose-dependently attenuated by specific inhibition of caspase-1 with z-YVAD-fmk (FIG. 14a). Furthermore, NO production was absent in microglial cultures from caspase-1^{-/-} mice after Aβ stimulation (FIG. 14b). Similarly, Aβ-induced TNF-α release was attenuated by caspase-1 inhibition (FIG. 14c), and was absent in caspase-1^{-/-} microglia (FIG. 14d). To ensure that the attenuation of cytokine release by caspase-1 inhibition was related to actions of IL-1β, and not to IL-1β-independent effects of caspase-1, we analyzed TNF-α and NO production in microglia deficient in IL-1 receptor. We found that the release of these factors was strongly reduced in IL-1 receptor^{-/-} microglia (FIG. 16a-b). Importantly, IL-1β levels remained unchanged compared to wildtype microglia cells (FIG. 16c), indicating that TNF-α and NO production depend on auto- and paracrine effects of IL-1β after caspase-1 activation and are unrelated to other effects of caspase-1. As control, TNF-α and NO levels were equally high in both cell lines after stimulation with the inflammasome-independent activator zymosan (FIG. 16a-b). These data show that release of important pro-inflammatory and neurotoxic factors depends on activation of caspase-1 and subsequent IL-1 signaling.

To confirm that upstream activation of the NALP3 inflammasome is necessary for caspase-1-dependent production of these factors, we measured NO release from macrophage cells lines of NALP3^{-/-} and ASC^{-/-} mice. As controls, we used cells from wild type mice and IPAF^{-/-} mice, a different inflammasome typically activated by intracellular bacterial pathogens²³. Consistent with the central role of the NALP3 inflammasome in Aβ-induced caspase-1 activation, we detected no significant NO release in cultures from macrophage cultures of NALP3^{-/-} or ASC^{-/-} mice, whereas strong NO production was measured in cultures from IPAF^{-/-} and wildtype mice (FIG. 19e). These data indicate that NALP3 inflammasome activation by Aβ is an important trigger for subsequent downstream activation of inflammatory and potentially cytotoxic mediators.

We therefore directly investigated whether caspase-1 contributes to neuronal dysfunction in vitro. We incubated Aβ with a mouse neuronal cell line and found only minimal morphological and quantitative effects (FIG. 14f, left column), as previously described in a similar system²⁴. In contrast, in mixed neuronal/microglial cell cultures with wild type microglial cells, Aβ induced widespread neuronal cell death (FIG. 14f, middle column), whereas no neurotoxic effects of Aβ were observed in mixed cultures with caspase-1^{-/-} mice microglia (FIG. 14f, right column).

Microglia have been shown to upregulate and secrete mononuclear phagocyte chemoattractants in response to Aβ²⁵, which contribute to additional accumulation of microglial cells around senile plaques. To investigate if the caspase-1 pathway is also involved in the upregulation of chemotactic cytokines, we stimulated wild type and caspase-1^{-/-} microglial cells with Aβ and determined mRNA induction of CCL-3/MIP1 a, CCL4/MIP1β and CXCL2/MIP2 using real-time quantitative PCR. Aβ time-dependently upregulated gene expression of these chemokines in wild type cells as described²⁵, whereas microglial cells deficient in caspase-1 failed to uregulate chemokine mRNA (FIG. 14g).

These results suggest that the NALP3 inflammasome, via activation of caspase-1, largely contributes to the pro-inflammatory, chemotactic and neurotoxic effects of Aβ mediated by microglia.

### Example 14

### Interleukin-1-mediated Pathways Contribute to Microglial Activation Induced by 13-Amyloid In Vivo

Finally, we investigated whether microglial chemotaxis and activation in vivo is mediated by inflammasome components, caspase-1 and the IL-1 pathway. To this end, we stereotactically injected fibrillar Aβ into the striatum of anesthetized adult mice (i.e., Wild type, ASC^{-/-}, caspase-1^{-/-}, IL-1R^{-/-}, or MyD88^{-/-} mice). This model has been used to study the inflammatory effects of Aβ in vivo and to investigate microglial function in models of Alzheimer's disease^{26,27}. As a control, non-fibrillar reverse Aβ peptide (revAβ) was injected into the contralateral hemisphere.

Immunohistochemistry of fixed brain sections stained with an antibody against the microglial and macrophage marker F4/80 (a marker for activated microglia and macrophages) 48 h after injection showed strong recruitment and chemotaxis of microglia around the injection site,whereas only few cells accumulated in the contralateral hemisphere injected with reverse Aβ (Fluorescence intensity of F4/80 staining was quantified as a measure of the number of recruited microglia and mononuclear phagocytes. Five consecutive sections in each mouse were quantified (mean±s.e.m.). Fluorescence intensities were normalized to wild type animals.

Next, we used different knockout mouse lines to explore the role of the inflammasome, caspase-1 activation, and IL-1 signaling in microglial recruitment to Aβ in vivo. First, we found that microglial recruitment and activation was significantly reduced in mice deficient in the inflammasome component ASC (ASC^{-/-}) Similarly, mice deficient in caspase-1 (caspase-1^{-/-}) also showed only very attenuated microglial activation after Aβ injection. Finally, recruitment and accumulation of microglia to Aβ was significantly reduced in mice deficient in IL-1 receptor (IL-11R^{-/-}) or the IL-1 receptor adaptor protein MyD88 (MyD88^{-/-}) Microglial activation in contralateral hemispheres injected with revAβ was very low in all groups.

These data demonstrate that, similar to microglia in vitro, microglial activation in vivo is strongly dependent on activation of the inflammasome and caspase-1, and subsequent initiation of the IL-1 pathway.

### References for Examples 9-14:

1. Weiner, H. L. & Frenkel, D. Immunology and immunotherapy of Alzheimer's disease. Nat Rev Immunol 6, 404-416 (2006).

2. Meyer-Luehmann, M. et al. Rapid appearance and local toxicity of amyloid-beta plaques in a mouse model of Alzheimer's disease. Nature 451, 720-724 (2008).

3. Simard, A. R., Soulet, D., Gowing, G., Julien, J. P. & Rivest, S. Bone marrow-derived microglia play a critical role in restricting senile plaque formation in Alzheimer's disease. Neuron 49, 489-502 (2006).

4. Itagaki, S., McGeer, P. L., Akiyama, H., Zhu, S. & Selkoe, D. Relationship of microglia and astrocytes to amyloid deposits of Alzheimer disease. J Neuroimmunol 24, 173-182 (1989).

5. Weggen, S. et al. A subset of NSAIDs lower amyloidogenic Abeta42 independently of cyclooxygenase activity. Nature 414, 212-216 (2001).

6. Schenk, D. et al. Immunization with amyloid-beta attenuates Alzheimer-disease-like pathology in the PDAPP mouse. Nature 400, 173-177 (1999).

7. Akama, K. T. & Van Eldik, L. J. Beta-amyloid stimulation of inducible nitric-oxide synthase in astrocytes is interleukin-lbeta- and tumor necrosis factor-alpha (TNFalpha)-dependent, and involves a TNFalpha receptor-associated factor- and

NFkappaB-inducing kinase-dependent signaling mechanism. J Biol Chem 275, 7918-7924 (2000).

8. Griffin, W. S. et al. Brain interleukin 1 and S-100 immunoreactivity are elevated in Down syndrome and Alzheimer disease. Proc Natl Acad Sci U S A 86, 7611-7615 (1989).

9. Blum-Degen, D. et al. Interleukin-1 beta and interleukin-6 are elevated in the cerebrospinal fluid of Alzheimer's and de novo Parkinson's disease patients. Neurosci Lett 202, 17-20 (1995).

10. Dinarello, C. A. A signal for the caspase-1 inflammasome free of TLR. Immunity 26, 383-385 (2007).

11. Maranon, F., Petrilli, V., Mayor, A., Tardivel, A. & Tschopp, J. Gout-associated uric acid crystals activate the NALP3 inflammasome. Nature 440, 237-241 (2006).

12. Mariathasan, S. et al. Cryopyrin activates the inflammasome in response to toxins and ATP. Nature 440, 228-232 (2006).

13. Dostert, C. et al. Innate Immune Activation Through NALP3 Inflammasome Sensing of Asbestos and Silica. Science (2008).

14. Amstad, P. A. et al. Detection of caspase activation in situ by fluorochrome-labeled caspase inhibitors. Biotechniques 31, 608-610, 612, 614, passim (2001).

15. Agostini, L. et al. NALP3 forms an IL-1β eta-processing inflammasome with increased activity in Muckle-Wells autoinflammatory disorder. Immunity 20, 319-325 (2004).

16. Fernandes-Alnemri, T. et al. The pyroptosome: a supramolecular assembly of ASC dimers mediating inflammatory cell death via caspase-1 activation. Cell Death Differ 14, 1590-1604 (2007).

17. Liu, Y. et al. LPS receptor (CD14): a receptor for phagocytosis of Alzheimer's amyloid peptide. Brain 128, 1778-1789 (2005).

18. Mueller-Steiner, S. et al. Antiamyloidogenic and neuroprotective functions of cathepsin B: implications for Alzheimer's disease. Neuron 51, 703-714 (2006).

19. Hook, V. Y., Kindy, M. & Hook, G Inhibitors of Cathepsin B Improve Memory and Reduce {beta}-Amyloid in Transgenic Alzheimer Disease Mice Expressing the Wild-type, but Not the Swedish Mutant, {beta} -Secretase Site of the Amyloid Precursor Protein. J Biol Chem 283, 7745-7753 (2008).

20. Buttle, D. J., Murata, M., Knight, C. G. & Barrett, A. J. CA074 methyl ester: a proinhibitor for intracellular cathepsin B. Arch Biochem Biophys 299, 377-380 (1992).

21. Xie, Z. et al. Peroxynitrite mediates neurotoxicity of amyloid beta-peptide1-42- and lipopolysaccharide-activated microglia. J Neurosci 22, 3484-3492 (2002).

22. Combs, C. K., Karlo, J. C., Kao, S. C. & Landreth, G. E. beta-Amyloid stimulation of microglia and monocytes results in TNFalpha-dependent expression of inducible nitric oxide synthase and neuronal apoptosis. J Neurosci 21, 1179-1188 (2001).

23. Mariathasan, S. & Monack, D. M. Inflammasome adaptors and sensors: intracellular regulators of infection and inflammation. Nat Rev Immunol 7, 31-40 (2007).

24. Li, M., Pisalyaput, K., Galvan, M. & Tenner, A. J. Macrophage colony stimulatory factor and interferon-gamma trigger distinct mechanisms for augmentation of beta-amyloid-induced microglia-mediated neurotoxicity. J Neurochem 91, 623-633 (2004).

25. El Khoury, J. B. et al. CD36 mediates the innate host response to beta-amyloid. J Exp Med 197, 1657-1666 (2003).

26. Weldon, D. T. et al. Fibrillar beta-amyloid induces microglial phagocytosis, expression of inducible nitric oxide synthase, and loss of a select population of neurons in the rat CNS in vivo. J Neurosci 18, 2161-2173 (1998).

27. El Khoury, J. et al. Ccr2 deficiency impairs microglial accumulation and accelerates progression of Alzheimer-like disease. Nat Med 13, 432-438 (2007).

28. Kanneganti, T. D. et al. Bacterial RNA and small antiviral compounds activate caspase-1 through cryopyrin/NALP3 . Nature 440, 233-236 (2006).

29. Ishii, K. J., Coban, C. & Akira, S. Manifold mechanisms of toll-like receptor-ligand recognition. J Clin Immunol 25, 511-521 (2005).

30. Frackowiak, J. et al. Ultrastructure of the microglia that phagocytose amyloid and the microglia that produce beta-amyloid fibrils. Acta Neuropathol 84, 225-233 (1992).

31. Cataldo, A. M., Barnett, J. L., Pieroni, C. & Nixon, R. A. Increased neuronal endocytosis and protease delivery to early endosomes in sporadic Alzheimer's disease: neuropathologic evidence for a mechanism of increased beta-amyloidogenesis. J Neurosci 17, 6142-6151 (1997).

32. Yang, A. J., Chandswangbhuvana, D., Margol, L. & Glabe, C. G. Loss of endosomal/lysosomal membrane impermeability is an early event in amyloid Abeta1-42 pathogenesis. J Neurosci Res 52, 691-698 (1998).

33. Kummer, J. A. et al. Inflammasome components NALP1 and 3 show distinct but separate expression profiles in human tissues suggesting a site-specific role in the inflammatory response. J Histochem Cytochem 55, 443-452 (2007).

34. Boraschi, D. & Tagliabue, A. The interleukin-1 receptor family. Vitam Horm 74, 229-254 (2006).

35. Shaftel, S. S. et al. Sustained hippocampal IL-1 beta overexpression mediates chronic neuroinflammation and ameliorates Alzheimer plaque pathology. The Journal of clinical investigation 117, 1595-1604 (2007).

36. Richard, K. L., Filali, M., Prefontaine, P. & Rivest, S. Toll-like receptor 2 acts as a natural innate immune receptor to clear amyloid beta 1-42 and delay the cognitive decline in a mouse model of Alzheimer's disease. J Neurosci 28, 5784-5793 (2008).

37. Tahara, K. et al. Role of toll-like receptor signalling in Abeta uptake and clearance. Brain 129, 3006-3019 (2006).

38. Franchi, L. et al. Cytosolic flagellin requires Ipaf for activation of caspase-1 and interleukin lbeta in salmonella-infected macrophages. Nature immunology 7, 576-582 (2006).

39. Kuida, K. et al. Altered cytokine export and apoptosis in mice deficient in interleukin-1 beta converting enzyme. Science 267, 2000-2003 (1995).

40. Halangk, W. et al. Role of cathepsin B in intracellular trypsinogen activation and the onset of acute pancreatitis. The Journal of clinical investigation 106, 773-781 (2000).

41. Moore, K. J. et al. A CD36-initiated signaling cascade mediates inflammatory effects of beta-amyloid. J Biol Chem 277, 47373-47379 (2002).

42. Severa, M., Coccia, E. M. & Fitzgerald, K. A. Toll-like receptor-dependent and -independent viperin gene expression and counter-regulation by PRDI-binding factor-1/BLIMPI. The Journal of biological chemistry 281, 26188-26195 (2006).

43. Lehnardt, S. et al. A mechanism for neurodegeneration induced by group B streptococci through activation of the TLR2/MyD88 pathway in microglia. J Immunol 177, 583-592 (2006).

44. Roberson, S. M. & Walker, W S Immortalization of cloned mouse splenic macrophages with a retrovirus containing the v-raf/mil and v-myc oncogenes. Cellular immunology 116, 341-351 (1988).

45. Blasi, E., Barluzzi, R., Bocchini, V., Mazzolla, R. & Bistoni, F. Immortalization of murine microglial cells by a v-raf/v-myc carrying retrovirus. J Neuroimmunol 27, 229-237 (1990).

46. Lois, C., Hong, E. J., Pease, S., Brown, E. J. & Baltimore, D. Germline transmission and tissue-specific expression of transgenes delivered by lentiviral vectors. Science 295, 868-872 (2002).

47. Qi, Y., Wang, J. K., McMillian, M. & Chikaraishi, D. M. Characterization of a CNS cell line, CAD, in which morphological differentiation is initiated by serum deprivation. J Neurosci 17, 1217-1225 (1997).

### Example 15

### Activation of the NALP3 Inflammasome by Cholesterol Crystallization

Atherosclerosis is fundamentally linked to an abnormally high cholesterol level in the blood (hypercholesterolemia), which ultimately leads to the deposition of excessive amounts of cholesterol in arterial walls. Excessive deposition of cholesterol leads to cholesterol supersaturation and, in turn, the production of cholesterol crystals in atherosclerotic lesions.

Experiments were conducted to test the hypothesis that cholesterol crystallization in atherosclerotic lesions is an endogenous danger signal which concomitant immune cell activation and induction of inflammation. Mouse macrophages were incubated with increasing amounts of cholesterol crystals (0, 0.25, 25, 100 or 400 μg/ml) and TNFα release into the supernatants was measured by ELISA according to standard methods. A significant increase in cytokine release corresponding to increasing levels of cholesterol crystals was observed.

These results demonstrated that cholesterol crystals were able to activate immune cells to release inflammatory cytokines, indicating that the formation of cholesterol crystals in atheromata are sufficient to trigger the inflammatory response via the inflammasome receptor complex in atherosclerosis.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The contents of all cited references (including literature references, patents, patent applications, and websites) that maybe cited throughout this application are hereby expressly incorporated by reference in their entirety for any purpose, as are the references cited therein. The practice of the present invention will employ, unless otherwise indicated, conventional techniques of immunology, molecular biology, cell biology, and drug manufacturing and delivery, which are well known in the art.

1. A method for treating an inflammatory disorder induced by particulate matter comprising administering to a subject in need thereof an effective amount of an inhibitor of a pH-activated protease, such that the particulate-induced inflammatory disorder is treated.

2. A method of inhibiting particulate-induced caspase-1 activation comprising administering to a subject in need thereof an effective amount of an inhibitor of a pH-activated protease, such that the particulate-induced caspase-1 activation is inhibited.

3. A method of inhibiting particulate-induced NALP3 -ASC-dependent caspase-1 activation comprising administering to a subject in need thereof an effective amount of an inhibitor of a pH-activated protease, such that the particulate-induced NALP3 -ASC-dependent activation is inhibited.

4. A method for treating an inflammatory disorder related to NALP3 -ASC-dependent caspase-1 activation comprising administering to a subject in need thereof an effective amount of an inhibitor of a pH-activated protease, such that the NALP3-ASC-dependent caspase-1 activation-induced inflammatory disorder is treated.

5. The method of claim 4, wherein the NALP3 -ASC-dependent caspase-1 activation is induced by particulate matter.

6. The method of claim 4, wherein the NALP3 -ASC-dependent caspase-1 activation is induced by a mutation in NALP3.

7. The method of claim 1-6, wherein the protease is a cathepsin.

8. The method of claim 7, wherein the cathepsin is cathepsin B.

9. The method of claim 1, wherein the disorder is a pulmonary disorder.

10. The method of claim 9, wherein the pulmonary disorder is selected from the group consisting of an acute lung injury, acute respiratory distress syndrome, asthma, silicosis, pneumonoconiosis, fiber-induced pulmonary fibrosis, asbestosis, chronic obstructive pulmonary disease, chronic bronchitis, emphysema and bronchiectasis.

11. The method of claim 1, wherein the disorder is acute joint inflammation.

12. The method of claim 11, wherein the disorder is gout or pseudogout.

13. The method of claim 1, wherein the disorder is atherosclerosis.

14. The method of claim 1, wherein the disorder is amyloidosis.

15. The method of claim 1, wherein the disorder is a reperfusion injury.

16. The method of claim 15, wherein the reperfusion injury is stroke or myocardial infarction.

17. The method of claim 1, wherein the disorder is transplant rejection.

18. The method of claim 17, wherein the transplant rejection is selected from the group consisting of acute rejection, chronic rejection or chronic allograft vasculopathy.

19. The method of claim 1, wherein the disorder is chronic non-healing of physical injury.

20. The method of claim 1, wherein the disorder is liver inflammation.

21. The method of claim 1, wherein the disorder is an autoimmune disease.

22. The method of claim 21, wherein the autoimmune disease is selected from the group consisting of systemic lupus erythematosus, rheumatoid arthritis or vasculitis.

23. The method of claim 22, wherein the vasculitis is immune complex vasculitis.

24. The method of claim 1, wherein the disorder is a neurodegenerative disease.

25. The method of claim 24, wherein the neurodegenerative disease is selected from the group consisting of Parkinson's disease, Alzheimer's disease, Amyotrophic Lateral Sclerosis and Creutzfeldt-Jakob disease.

26. The method of claim 1, wherein the disorder is a periodic fever syndrome.

27. The method of claim 26, wherein the periodic fever syndrome is selected from the group consisting of Familial Mediterranean fever; TNF receptor 1-associated periodic syndrome; Hyper-IgD syndrome; Periodic fevers with Aphthous stomatitis, Pharyngitis and Adentitis syndrome; pyogenic sterile arthritis, pyoderma gangrenosum and acne syndrome; Blau syndrome; and a cryopyrin-associated periodic syndrome.

28. The method of claim 27, wherein the cryopyrin-associated periodic syndrome is selected from the group consisting of familial cold autoinflammatory syndrome, Muckle-Wells syndrome and neonatal onset multisystem inflammatory disorder.

29. The method of claim 1, wherein the disorder is a blockage of the ureter.

30. The method of claims 1, wherein the particulate matter is selected from the group consisting of a crystal or a fiber.

31. The method of claim 30, wherein the crystal comprises monosodium urate (MSU), aluminum salt, silica, calcium pyrophosphate dehydrate (CPPD), cholesterol and beta amyloid.

32. The method of claim 31, wherein the fiber is asbestos or a nonasbestiform mineral fiber.

33. The method of claim 1, wherein the particulate matter is selected from the group consisting of apoptotic cells, necrotic cells, immune complexes, minimally modified LDL, aggregated peptides and aggregated proteins.

34. The method of claim 1, wherein the particulate matter is a kidney stone.

35. The method of claim 33, wherein the aggregated peptides comprise amyloid plaques.

36. The method of claim 33, wherein the aggregated proteins comprise aggregated alpha-synuclein.

37. The method of claim 33, wherein the aggregated proteins comprise copper-zinc superoxide dismutase and Bcl-2.

38. The method of claim 33, wherein the aggregated peptides comprise prions.
